(19) United States
(12) Patent Application Publication
(10) Pub. No.: US 2008/0108869 A1
(43) Pub. Date: May 8, 2008

(54) Optical surgical device and methods of use

(75) Inventors: Gerald J. Sanders, Sonoma, CA (US); Vivek Sikri, Cambridge, MA (US)

Correspondence Address: MINTZ LEVIN COHN FERRIS GLOVSKY & POPEO, ONE FINANCIAL CENTER, BOSTON, MA, 02111, US

(73) Assignee: Femsuite LLC, San Francisco, CA (US)

(21) Appl. No.: 11/975,409

(22) Filed: Oct. 19, 2007

Related U.S. Application Data
(60) Provisional application No. 60/853,161, filed on Oct. 20, 2006.
(60) Provisional application No. 60/878,892, filed on Jan. 4, 2007.
(60) Provisional application No. 60/903,583, filed on Feb. 26, 2007.
(60) Provisional application No. 60/921,925, filed on Apr. 4, 2007.
(60) Provisional application No. 60/925,486, filed on Apr. 20, 2007.
(60) Provisional application No. 60/933,233, filed on Jun. 4, 2007.

(51) Int. Cl. A61B 1/04

(52) U.S. Cl. 600/109

(57) ABSTRACT

An optical device includes a shaft, a handle and a camera assembly. The handle is coupled to the shaft at a first end, and the camera assembly is coupled to the shaft at a second end. Camera circuitry and software may be provided in the shaft and the handle, so that, in one embodiment, the device may be constructed with reusable portions of the camera circuitry and software. In another embodiment, the device may be provided as a single piece, that may be discarded or sterilized after use.

### CROSS-REFERENCE TO RELATED APPLICATIONS

This application claims priority to U.S. provisional patent application Nos. 60/853,161, filed Oct. 20, 2006, 60/878,892, filed Jan. 4, 2007, 60/903,583, filed Feb. 26, 2007, 60/921,925, filed Apr. 4, 2007, 60/925,486, filed Apr. 20, 2007, and 60/933,233, filed Jun. 4, 2007, all of which are hereby incorporated by reference herein in their entireties.

### FIELD OF THE INVENTION

The present invention relates to apparatus and methods for imaging body tissue during medical procedures. More particularly, the present invention relates to apparatus and methods that provide endoscopic viewing of the female genital tract during gynecological procedures.

### BACKGROUND OF THE INVENTION

A number of gynecological procedures require visual inspection of the genital tract of a female patient, which is often performed with the use of an endoscope, or hysteroscope. Conventional endoscopes are often constructed from a rigid body and oftentimes those rigid bodies include fluid conduits.

For example, colposcopy is a diagnostic procedure, in which a lighted binocular microscope called a colposcope is utilized to examine an illuminated, magnified view of the vulva, vagina, and cervix. Most women undergo colposcopic examination to investigate abnormalities in their pap smears, or to assess diethylstilbestrol (DES) exposure in utero, HIV infection or immunosuppression. The enlarged view provided by a colposcope enables a clinician to visually distinguish normal from abnormal appearing tissue and take directed biopsies for pathological examination.

Colposcopy is performed with the patient in the dorsal lithotomy position, in which the patient lies with her legs in stirrups and her buttocks close to the lower edge of the examining table. A speculum is placed in the vagina after the vulva is examined for any suspicious lesions, and an acetic acid solution (e.g., Lugol's or Schiller's) is applied to the cervix to improve visualization and to help the clinician assess whether a change in color or in the vascular pattern of the patient are indicative of abnormalities. After a complete examination, the clinician determines the areas with the highest degree of visual abnormality and obtains biopsies from these areas using a long biopsy instrument.

Colposcopy is an expensive procedure that requires a dedicated instrument, the colposcope, an a specially trained clinician. While colposcopy is considered a preferred procedure for diagnosing cervical abnormalities, it also has come drawbacks. The cost of the colposcope and of the clinical training required to perform it limit application. Additionally, the colposcope is a bulky instrument, usable only in dedicated clinical settings, and provides no view of the uterus. Due to the nature of the colposcope, separate instruments must be employed for taking biopsies and, when required, for endocervical curettage (ECC).

The uterine cavity may be examined by hysteroscopy, which is a diagnostic procedure that enables a clinician to diagnose intrauterine pathology and which may provide a method for surgical intervention (operative hysteroscopy).

Hysteroscopy is performed with an endoscopic device, called a hysteroscope. Some hysteroscopes include a stiff shaft coupled to a handle, a vision member at the tip of the shaft connected to fiber optics and to a video system, and a channel for delivering a distention medium. Because the uterus is a potential cavity, it is first distended either with a fluid (saline, sorbitol, or a dextrane solution) or a gas (CO₂), and the stiff shaft carrying the vision member is introduced in the uterus through the cervical canal.

Different types of hysteroscopes may be used for different gynecological interventions. While the hysteroscope is typically a viewing device only, an operative hysteroscope includes a working channel that allows specialized instruments to enter the uterine cavity and perform surgery, and a resectoscope is a variation of the hysteroscope that contains an electric loop for resecting a submucous leiomyoma.

Hysteroscopy has been found useful to treat a variety of uterine conditions, such as polyps, leiomyomata, Asherman syndrome, gynecologic bleeding, and uterine malformations, but occasionally a uterine perforation occurs when the stiff shaft breaches the wall of the uterus leading to bleeding and to damage to other organs. Another drawback of known hysteroscopes is limited maneuverability, due to the rigidity of the shaft that makes it difficult to maneuver the instrument within the patient's genital system. Still another drawback relates to the use of fiber optics, which are manufactured from glass that breaks under bending stress, requiring frequent maintenance of the hysteroscope with consequent downtime and costs. Additionally, in known hysterocopes the camera, saline channel, and working channel all have distal openings at the distal tip of the shaft, causing an increase in the diameter of the tip and making the instrument more invasive to the patient. A corresponding decrease in channel diameters decreases the efficiency of the instrument and makes it more difficult to clean and sterilize.

Attempts have been made to remedy these drawbacks of conventional hysteroscopes. For example, U.S. Pat. No. 4,836,189 to Allred, III et al. describes a video hysteroscope having an elongated flexible insertion tube containing a video member at its distal end, as well as a channel for a surgical laser fiber and a saline channel that emits a continuous stream of saline solution. An articulation section joins the viewing head to a flexible tubular member.

U.S. Pat. No. 5,823,940 to Newman discloses a sheath that receives an endoscope. In that device, the endoscope includes a bundle of fiber optics that is slid into a lumen in the sheath. The sheath is flexible and includes additional fluid conduits. After a procedure is performed with the sheath, the endoscope is removed from the sheath, which is then discarded.

U.S. Publication No. 2005/0288551 to Callister et al. discloses an endoscopic assembly having a flexible hysteroscope and an outer sheath disposed about a length of the shaft of the hysteroscope. An inflatable balloon seals the assembly within a body lumen or cavity.

A drawback of these devices is that although some of them contain disposable components, the hysteroscope and an associated eyepiece still require cleaning and sterilization, contain fragile fiber optics, and have tips with sizes that make the instruments uncomfortable or even painful when traveling through the cervical canal and into the uterus, and correspondingly limit the diameters of the lumens in the instrument. Another drawback is that the light colors provided by some of these instruments are within a limited palette, while different types of anomalies are better viewable with different light combinations.

It would be therefore be desirable to provide improved apparatus and methods for inspecting body tissues that, in various embodiments, remedy some or all of the aforementioned drawbacks of previous optical devices.

### SUMMARY OF THE INVENTION

Some embodiments of the present invention provide an optical device for inspecting body tissue. In some embodiments, the apparatus and methods methods may utilize flexible tips, so to enable the examination of tortuous anomalies and minimize the risks of perforations.

In some embodiments, the apparatus and methods do not require optical fibers, thereby enhancing flexibility and versatility of the device.

In still other embodiments, the apparatus and methods provide a plurality of channels and light colors within a narrow instrument tip.

In some embodiments, the apparatus and methods require little or no sterilization. For example, the apparatus may be provided sterile and may be used only once.

Some embodiments provide apparatus and methods for ob-gyn examinations that enable a detailed examination of the uterine cavity. The apparatus and methods may be implementable in any setting, even in field situations.

In some embodiments, an optical device is provided that includes a disposable portion and a reusable portion, which may simplify cleaning and sterilization.

In still other embodiments, an optical device is provided that is shapeable by a user so that it may be shaped into a desired curvature.

Some embodiments of the present invention provide an optical apparatus including a shapeable shaft, a removable portion, such as a removable handle or cartridge, coupled to a first end of, or within, the shapeable shaft and a camera coupled to the second end of the shaft.

In an embodiment, the removable portion houses camera circuitry and/or a light source (e.g., a high-powered light source such as a high-powered light-emitting diode (LED)) and may be reused. Including the removable portion allows the shaft to be discarded after use while the removable portion may be reused. The configuration of the housing provides easy cleaning and sterilization when necessary, if at all. In some embodiments, the optical device is manufactured from low-cost components that make it economical to dispose of at least a portion of the device after use instead of sterilizing the device.

The camera may be configured to move relative to the shaft such as, for example, by a hinged and/or rotating coupling. The relative movement may allow a user to orient the camera as desired to image a particular area as desired or to provide clearance, or access, to a working channel and/or one or more fluid conduits included in the shaft. In some embodiments, a working channel may be provided that has a non-circular cross-section, which may allow the channel to serve simultaneously as a fluid conduit.

In some embodiments, an optical apparatus is provided that includes a housing including a shaft portion, and a handle portion extending from a proximal end of the shaft portion and defining a cavity that contains an output connector. A camera assembly that includes a camera is coupled to a distal end of the shaft. A removable (e.g., reusable) cartridge is provided that is receivable within the cavity and that includes, for example, an input connector matable with the output connector, an image processing engine (e.g., digital signal processor), a storage module, a power source, and an output module. The output module may be a wireless output module, a analog (e.g., NTSC/PAL) output module, and/or a USB output module.

Alternatively or additionally, the removable cartridge may include a light-emitting diode (e.g., high-powered LED). An optical couple (e.g., one or more optical fibers and/or couples between the fibers) may be provided for carrying light from the light-emitting diode at least partially through the shaft.

Alternatively or additionally, the removable cartridge may include an accelerometer in communication with the image processing engine. For example, in response to the accelerometer sensing rotation of the camera assembly, the accelerometer may cause the image processing engine to rotate an image from the camera.

In some embodiments, a method of ob-gyn examination is provided that includes inserting a shaft of an optical apparatus into the vaginal canal of a patient. A camera assembly that includes a camera may be provided that is coupled to a distal end of the shaft. The camera assembly may be rotated radially relative to the shaft to expose a working channel. At least one image from the camera may be displayed to allow for visual examination of at least one of the tissue and vascular structure of one or more of the cervix, the vagina, and the vulva. In some embodiments, at least one surgical instrument may be inserted into the vaginal canal through the working channel. In some embodiments, the rotating of the camera assembly may also expose light from a light-emitting diode. For example, the LED may be turned on in response to the rotating. In some embodiments, at least one image may be corrected for the rotating prior to the displaying.

In other embodiments, a method of ob-gyn examination is provided that includes inserting a shaft of an optical apparatus into the vaginal canal of a patient and capturing at least one image with a camera coupled to a distal end of the shaft without the use of fiber optics. The camera may be moved, without rotation, relative to the shaft to change the field of view of the camera. For example, in some embodiments, the moving without rotating may include moving the camera relative to the shaft about a hinge, where the hinge comprises an axis that is perpendicular to an axis of the shaft. In other embodiments, the moving without rotating may include sliding the camera outwardly along a ramp interface between the camera and the distal end of the shaft. In still other embodiments, the moving without rotating may include coupling an arm of the camera to a rail formed in the shaft and sliding the arm through a predetermined path defined by the rail to move the camera outwardly from shaft.

In some embodiments, an optical apparatus is provided that includes a shaft having a first end and a second end and a working channel extending longitudinally through at least a portion of the shaft to the second end. A handle may be provided that is coupled to the first end of the shaft. A camera assembly comprising a camera may also be provided. A flexible circuit may be provided that is coupled to the camera and to the second end of the shaft. A control member may be provided that is coupled to the camera assembly and to the second end of the shaft, where rotation of the control member causes rotation of the camera assembly relative to the shaft from a first configuration in which the camera assembly blocks an opening of the working channel to a second, rotated configuration in which the working channel is exposed. For example, in the first configuration the camera assembly may be aligned concentrically with the shaft and in the second configuration the camera assembly may be spaced radially from the shaft.

In some embodiments, the control member may include a pin having a longitudinal axis that is parallel to a longitudinal axis of the shaft.

In some embodiments, the control member may extend to at least a proximal end of the shaft.

Alternatively or additionally, in some embodiments the shaft may include a generally crescent-shaped lumen positioned concentrically with respect to the control member. The optical apparatus may further include a second member configured to traverse the crescent-shaped lumen responsive to rotation of the control member coupled to the camera assembly. In some embodiments, the flexible circuit may be positioned at least partially within the crescent-shaped lumen such the flexible circuit remains generally adjacent to the second member during the translation of the second member.

In some embodiments, the camera assembly may include a proximal circumferential step. The shaft may also include a circumferential step included on the distal end of the shaft. In the first configuration, the circumferential step of the camera assembly may be configured to mate with the circumferential step of the shaft and in the second configuration the circumferential step of the camera assembly may be adapted to contact an outer surface of the shaft.

In still other embodiments, an optical apparatus may be provided that includes a shaft having a first end and a second end, a handle coupled to the first end of the shaft, a camera assembly comprising a camera and a flexible circuit extending between the camera assembly and to the second end of the shaft. The shaft and the camera assembly may be coupled by a hinge joint and the flexible circuit may extend through the hinge joint.

For example, in some embodiments, the hinge joint may include a cylindrical projection that is received by, and secured in place by frictional forces from, a cylindrical socket. A lumen for receiving the flexible circuit may extend through the cylindrical projection. In some embodiments, the lumen may have a tapered opening on the cylindrical projection.

In other embodiments, the optical apparatus may include a hinge pin that extends laterally through the hinge joint.

### BRIEF DESCRIPTION OF THE DRAWINGS

The foregoing summary is only illustrative of the inventions disclosed herein. Further features of the invention, its nature and various advantages will be more apparent from the accompanying drawings and the following detailed description of the preferred embodiments, in which:

FIG. 1 is a side view of an embodiment of the assembled optical device in accordance with the present invention;

FIG. 2 is a cross-sectional view of a portion of the optical device of FIG. 1;

FIG. 3 is an end view of the embodiment of FIG. 2;

FIG. 4 is a side view of an exemplary camera and flex circuit assembly that may be used in the optical device of the present invention;

FIG. 5 is an exploded view of a camera assembly of the optical device of FIG. 1;

FIG. 6 is a perspective view of the end portion of the camera assembly of FIG. 5;

FIG. 7 is a cross-sectional view of a portion of an alternative embodiment of an optical device;

FIG. 8 is a cross-sectional view of a portion of another alternative embodiment of an optical device;

FIG. 9 is a cross-sectional view of a portion of another alternative embodiment of an optical device;

FIG. 10 is an end view of the portion shown in FIG. 9;

FIG. 11 is a cross-sectional view of a portion of another alternative embodiment of an optical device;

FIG. 12 is an end view of the portion shown in FIG. 11;

FIG. 13 is a cross-sectional view of a portion of another alternative embodiment of an optical device;

FIG. 14 is an end view of the shaft shown in FIG. 13;

FIG. 15 is a cross-sectional view of an embodiment of the interface between a shaft and a removable handle of an optical device in accordance with the present invention;

FIG. 16 is a cross-sectional view of another embodiment of the interface between a shaft and a removable handle of an optical device;

FIG. 17 is a cross-sectional view of another embodiment of the interface between a shaft and a removable handle of an optical device;

FIG. 18 is a cross-sectional view showing a protective sleeve covering a removable handle of an optical device;

FIG. 19 is a side view of another embodiment of the optical device in accordance with the present invention;

FIG. 20 is a perspective view of a portion of another embodiment of the optical device in accordance with the present invention;

FIG. 21 is a side view of the portion of the embodiment of FIG. 20;

FIG. 22 is a side detail view of a portion of the device of FIG. 20;

FIG. 23 is a partial cross-sectional perspective view of a portion of the device of FIG. 20;

FIG. 24 is perspective view of a shaft included in the device of FIG. 20;

FIG. 25 is a side view of a portion of another embodiment of the optical device in accordance with the present invention;

FIG. 26 is a side view of a distal end portion of another embodiment of the optical device in accordance with the present invention;

FIG. 27 is a cross-sectional view of the device of FIG. 26;

FIG. 28 is a perspective view of a shaft tip included in the device of FIG. 26;

FIG. 29 is a perspective view of a camera housing included in the device of FIG. 26;

FIG. 30 is a cross-sectional view of a distal end portion of another embodiment of the optical device;

FIG. 31 is another cross-sectional view of the embodiment of FIG. 30;

FIG. 32A is a top view of an embodiment of a flexible printed circuit included in the optical device in accordance with the present invention;

FIGS. 32B and 32C are perspective views of another embodiment of a foldable, flexible printed circuit that may be provided within the optical device in accordance with the present invention;

FIG. 33 is a top view of a distal end portion of another embodiment of the optical device in a first, insertion configuration in accordance with the present invention;

FIG. 34 is a top view of the distal end portion of the embodiment of FIG. 33 in a second configuration;

FIG. 35 is an end view of the distal end portion of the embodiment of FIG. 33 in the second configuration;

FIG. 36 is a perspective view of a portion of a camera assembly housing included in the embodiment of FIG. 33;

FIG. 37 is a perspective view of a shaft tip included in the embodiment of FIG. 33;

FIG. 38 is an end view of the shaft tip included in the embodiment of FIG. 33;

FIG. 39 is a cross-sectional view taken along line A-A of FIG. 38;

FIG. 40 is a perspective view of a portion of the camera assembly housing coupled to the shaft tip included in the embodiment of FIG. 33;

FIG. 41 is a cross-sectional view taken along line B-B of FIG. 33 with a camera assembly shown in phantom;

FIG. 42 is a cross-sectional view taken along line C-C of FIG. 34 with a camera assembly shown in phantom;

FIG. 43 is a perspective view of a distal end portion of another embodiment of the optical device in a first, insertion configuration in accordance with the present invention;

FIG. 44 is a perspective view of the distal end portion of the embodiment of FIG. 43 in a second configuration;

FIG. 45 is another perspective view of the distal end portion of the embodiment of FIG. 43 in the second configuration;

FIG. 46 is a cross-sectional view of an embodiment of a shaft that may be included in the optical device;

FIG. 47 is a cross-sectional view of another embodiment of a shaft that may be included in the optical device;

FIG. 48 is a cross-sectional view of an embodiment of a shaft that may be included in the optical device;

FIG. 49 is a cross-sectional view of an embodiment of a shaft that may be included in the optical device of;

FIG. 50 is a cross-sectional top view of a distal end portion of another embodiment of the optical device in a first, insertion configuration in accordance with the present invention;

FIG. 51 is a cross-sectional top view of the distal end portion of the embodiment of FIG. 50 in a second configuration;

FIG. 52 is a cross-sectional top view of a distal end portion of another embodiment of the optical device in a first, insertion configuration in accordance with the present invention;

FIG. 53 is a cross-sectional top view of the distal end portion of the embodiment of FIG. 52 in a second configuration;

FIG. 54 is a cross-sectional top view of a distal end portion of another embodiment of the optical device in a first, insertion configuration in accordance with the present invention;

FIG. 55 is a cross-sectional top view of the distal end portion of the embodiment of FIG. 54 in a second configuration;

FIG. 56 is a perspective view of a distal end portion of another embodiment of the optical device in a first, insertion configuration in accordance with the present invention;

FIG. 57 is a side view of the distal end portion of the embodiment of FIG. 56 in a second configuration;

FIG. 58 is a cross-sectional perspective view of a distal end portion of another embodiment of the optical device in a first, insertion configuration in accordance with the present invention;

FIG. 59 is a cross-sectional side view of the distal end portion of the embodiment of FIG. 58 also in the first, insertion configuration;

FIG. 60 is a cross-sectional top view of the distal end portion of another embodiment of the optical device;

FIG. 61 is a cross-sectional top view of the distal end portion of another embodiment of the optical device;

FIG. 62 is a side view of another embodiment of the assembled optical device in accordance with the present invention;

FIG. 63 is a perspective view of another embodiment of the assembled optical device in accordance with the present invention;

FIG. 64 is a cross-sectional view of a working channel closure mechanism and camera control feature in a closed configuration;

FIG. 65 is a cross-sectional view of the closure mechanism of FIG. 64 in an open configuration;

FIG. 66 is a cross-sectional view of another embodiment of a working channel closure mechanism and camera control feature in a closed configuration;

FIG. 67 is a cross-sectional view of the closure mechanism of FIG. 66 in an open configuration;

FIG. 68 is a partial cross-sectional view of another embodiment of a working channel closure mechanism and camera control feature in a closed configuration;

FIG. 69 is a schematic diagram of a electronic switch that may be incorporated into a camera control feature;

FIG. 70 is a side view of an instrument holder that may be included in the optical device in accordance with the present invention;

FIG. 71 is an embodiment of an elongate body member of a shaft that may be included in the optical device in accordance with the present invention;

FIG. 72 is a side view of a portion of another embodiment of the optical device in accordance with the present invention;

FIG. 73 is a cross-sectional view of another embodiment of the optical device in accordance with the present invention;

FIG. 74 is a schematic diagram of a removable cartridge portion of the optical device of FIG. 73, and a device for receiving signals transmitted by the optical device, in accordance with some embodiments of the present invention;

FIG. 75 is a side view of a control feature locking feature of an optical device in accordance with the present invention;

FIG. 76 is a side view of position indicia of another control feature of an optical device in accordance with the present invention;

FIGS. 77A and 77B are top views a shaft that includes a semi-rigid portion coupled to a rigid portion for facilitating deflection of a distally-positioned camera according to some embodiments of the present invention;

FIG. 78 shows an embodiment of a semi-circular knob for connecting to pull wires that traverse the shaft of FIGS. 77A and 77B;

FIG. 79 shows an accelerometer and a digital signal processor for correcting the orientation of images received from a camera distally-positioned relative to a handle housing according to some embodiments of the present invention; and

FIG. 80 is a simplified view of an optical device that includes a high-powered light-emitting diode (“LED”) within a reusable part of the device according to some embodiments of the present invention.

### DETAILED DESCRIPTION OF THE INVENTION

The present invention is directed to an optical device, or endoscope, for use during medical procedures to view body tissue. The device may be constructed from detachable portions that may be reused or discarded as desired so that cleaning and sterilization may be simplified.

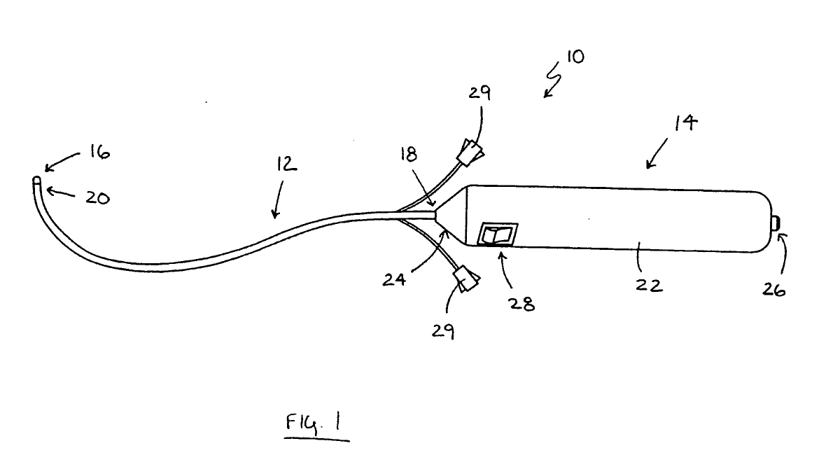

Referring to FIG. 1, an embodiment of the optical device will be described. Optical device 10 generally includes a shaft 12, handle 14 and camera assembly 16. Shaft 12 extends between handle 14 and camera assembly 16 and may be generally flexible. Shaft 12 may be configured so that after it is bent by a user it retains the bent configuration but may be easily reconfigured if desired. Such behavior is referred to herein as “shapeability,” and may be achieved by embedding stiffening members, such as stiffening wires, or blades, in the shaft as will be described in greater detail below. Fluid connectors 29 may also be provided so that fluid may be injected or aspirated through fluid conduits in shaft 12. Additionally, it will be appreciated that shaft 12 may be any length required to inspect any desired target tissue. For example, in embodiments used for gynecological inspection, the length of shaft 12 is generally in the range of 15-20 inches. In other embodiments, shaft 12 may be rigid.

In the embodiment shown in FIG. 1, first end 18 of shaft 12 is removably coupled to an end of handle 14 so that shafts having different configurations may be utilized with a single handle and/or so that a disposable shaft may be utilized with a reusable handle. Shaft 12 may be configured to provide a plurality of configurations with or without fluid conduits and/or working channels (i.e., a channel that allows a separate surgical device to be advanced through shaft 12). In addition, shaft 12 may be disposable so that it is discarded after use rather than requiring cleaning and sterilization. It should also be appreciated that all of optical device 10 may be disposable if desired.

Shaft 12 may be constructed from any material known in the art such as, for example, latex or silicone rubbers. The shapeability of shaft 12 may be provided by one or more bendable wires or blades, or a gooseneck embedded in shaft 12 or coupled to shaft 12.

Additionally, the shaft of the optical device may have any cross-sectional shape. For example, the shaft may be circular or polygonal in cross-section as desired. It should also be appreciated that controls, such as a slider switch, dial, or knob, may be included at first end 18 of shaft 12 to control movement of camera assembly 16 relative to shaft 12. As will be described in greater detail below, indicia may be provided with the controls to indicate the position of camera assembly 16 relative to shaft 12. Furthermore, the controls may be configured so that the position of camera assembly 16 relative to shaft 12 may be temporarily locked.

Handle 14 generally houses support electronics and controls for camera assembly 16. Handle 14 includes housing 22, input connector 24, output connector 26 and controls 28. In the present embodiment, housing 22 is generally cylindrical and coaxial with shaft 12. It should be appreciated that the handle may include contours that provide a more comfortable grip by a user and the handle may extend from first end 18 of shaft 12 at any angle.

Input connector 24 is configured to provide an electrical connection between electronics housed in shaft 12 and camera assembly 16 and the electronics of housing 22. Output connector 26 is configured to provide an electrical interface between peripheral support components such as a monitor, computer and/or power source.

Input connector 24 and output connector 26 may be any type of connector known in the art that provides the number of electrical conductors required to pass the desired electrical signals. For example, the connectors may be headphone jacks or multi-pin connectors. Alternatively, output connector 26 may be a wire pigtail that extends out of handle 14 and includes a connector at the end of the pigtail. Housing 22 may be constructed from any desired material such as plastic or metal.

Controls 28 provide a user the ability to control various attributes of optical device 10. Those attributes may include camera zoom, camera focus, camera position (e.g., orientation of the optical axis through bending and/or rotation of the camera relative to the shaft), and lighting attributes such as color and/or brightness, etc. It should be appreciated that color control may be achieved by electronic adjustment of an image sensor chip and/or by optical techniques. Controls 28 may include any type and number of control devices such as toggle switches, sliding switches, push buttons, dials and knobs. Controls 28 may also provide control over the flexing of shaft 12 or movement of camera assembly 16 relative to shaft 12, such as by a mechanical linkage, and controls 28 may be included on handle 14 and/or shaft 12 as desired.

Second end 20 of shaft 12 is coupled to camera assembly 16. Camera assembly 16 is preferably coupled to second end 20 of shaft 12 so that camera assembly 16 and shaft 12 are combined to form a single disposable unit. Camera assembly 16 provides image capturing and lighting capabilities. In an embodiment, the camera assembly is configured to provide a field of view of 70-100 degrees, to have a focal range between 5 mm and 50 mm, preferably between 30 mm and 50 mm, and a focal length of approximately 0.77 mm, and to be of TV quality, providing less than 29% TV distortion. It will be appreciated that a camera assembly may be included that provides any desired characteristics. The diameter of second end 20 is preferably approximately 4.5 mm.

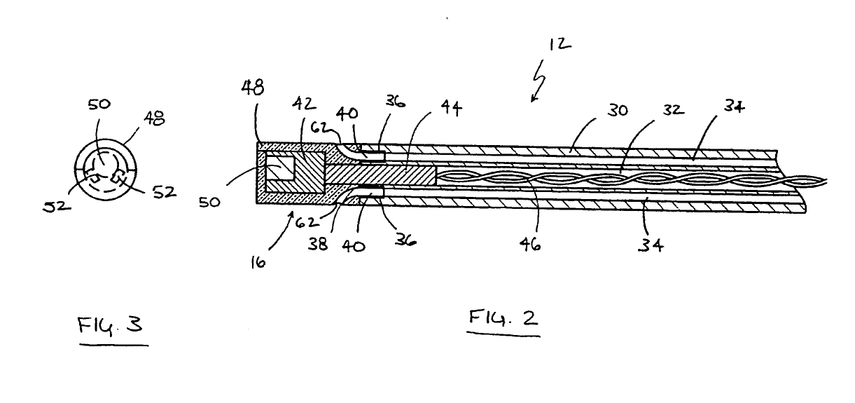

Referring to FIG. 2, shaft 12 will be described. Shaft 12 includes elongate body member 30 that extends between handle 14 and camera assembly 16. Body member 30 is generally tubular and defines lumen 32 that extends longitudinally through the entire length of body member 30. Body member 30 also defines a plurality of fluid conduits 34 that extend longitudinally through body member 30. Fluid conduits 34 are configured to carry fluid that may be used to clear the view of optics 50 included in camera assembly 16 and/or to distend the uterus during a gynecological examination to prevent the uterus from collapsing during examination. For example, fluid conduits may provide introduction and/or aspiration of saline, carbon dioxide (CO2) or any other fluid.

Fluid may be fed into or drawn out of the fluid conduits by gravity, a pump, a compressor, and/or a pressurized fluid source; for example, a full saline bag may be raised to insert the saline into the uterus via gravity and/or with the aid of a pump. If a pump is utilized it may be integrated into the optical device or external to the device as desired. It should be appreciated that fluid conduits 34 may be located within body member 30 and configured so that when not in use, fluid conduits 34 may be collapsed radially inward. For example, a collapsing fluid conduit may be constructed from the same material as the extruded body member, which is preferably an elastic material. The wall thickness adjacent fluid conduit 34 may be maintained thin so that the fluid conduit 34 inflates when pressurized and collapses when pressure is removed or a vacuum is drawn.

Second end 20 of shaft 12 provides an interface with camera assembly 16. Second end 20 includes fluid ports 36 that are coupled to fluid ports 40 included in camera assembly 16 and a lumen opening 38 that allows a portion of camera assembly 16 to be inserted into lumen 32 of body member 30.

Referring to FIGS. 2-6, camera assembly 16 includes camera head 42 that is electrically coupled to flex circuit 44 and a plurality of conductors, such as wires 46. Camera head 42 is at least partially enclosed in a distal camera housing assembly 48, which may be constructed from a transparent (i.e., optically clear) material.

Camera head 42 includes optic 50 and light sources 52. Optic 50 may be any suitable optic known in the art for example, optic 50 may be a charge coupled device (CCD) or complementary metal oxide semiconductor (CMOS) type image sensor. Similarly, light sources 52 may be any suitable light sources that will not substantially increase the diameter of shaft 12 or housing 48. For example, light sources 52 may be white and/or colored light-emitting diodes (LED). In an embodiment, white LEDs are incorporated as light sources 52 and may have an emission of 0-100 Lux or brighter.

Camera controls may be provided so that the white balance may be adjusted to provide a desired image. For example, it has been found that it is often preferable to enhance the green component in images taken during gynecological exams so either green LEDs or a white balance mode configured to enhance the green light component may be employed. The white balance mode may be provided by appropriate circuitry and software that may be housed in handle 14 or included as part of camera assembly 16. Additionally, controls such as a dial may be provided on handle 14 so that a user may adjust brightness and/or to make the image more or less green as they desire. Still further, controls and/or software may be provided that rotate the image in embodiments in which the camera assembly rotates along an axis generally parallel to the longitudinal axis of the shaft.

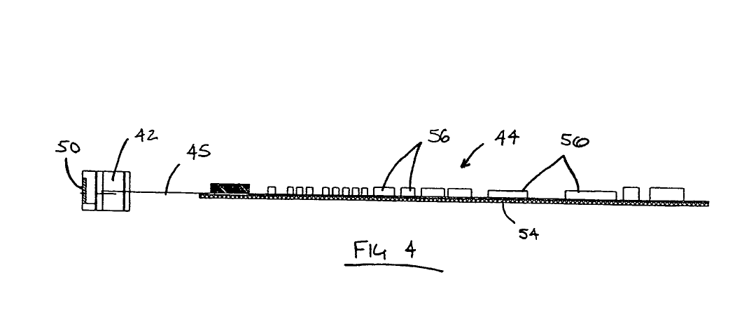

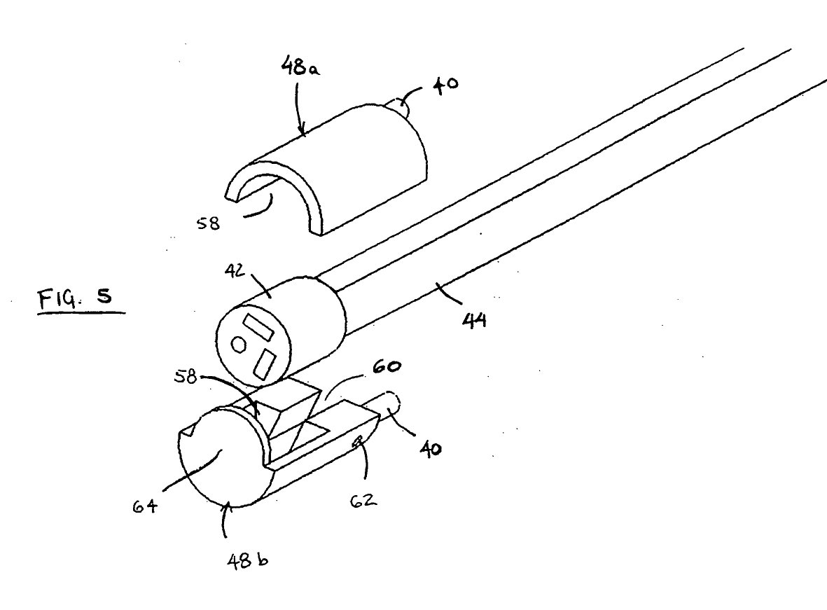

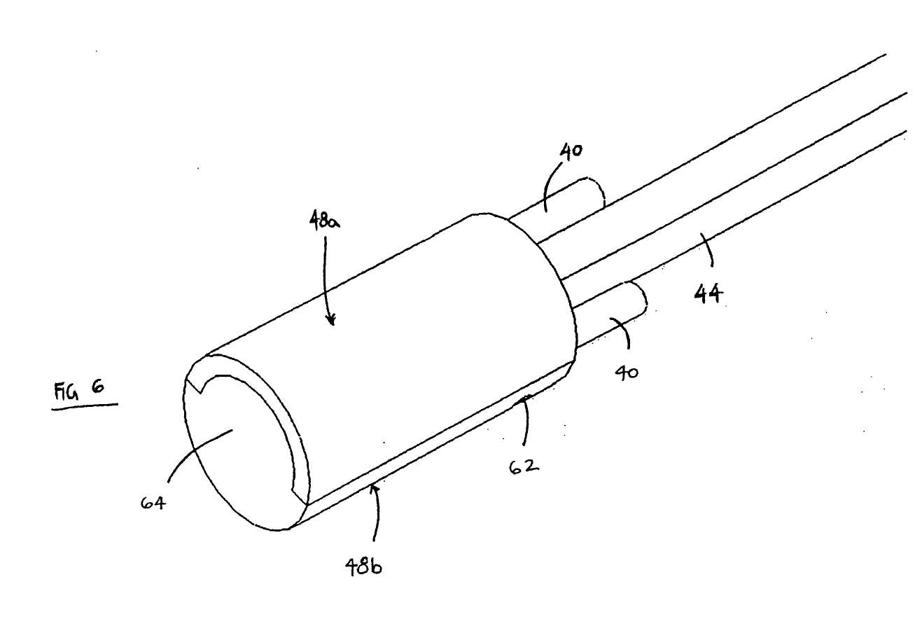

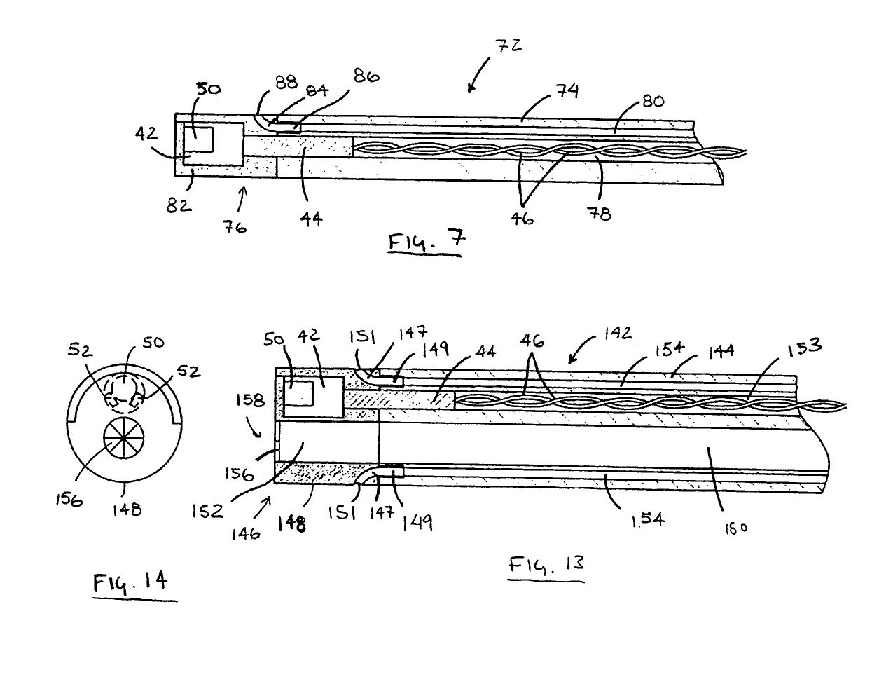

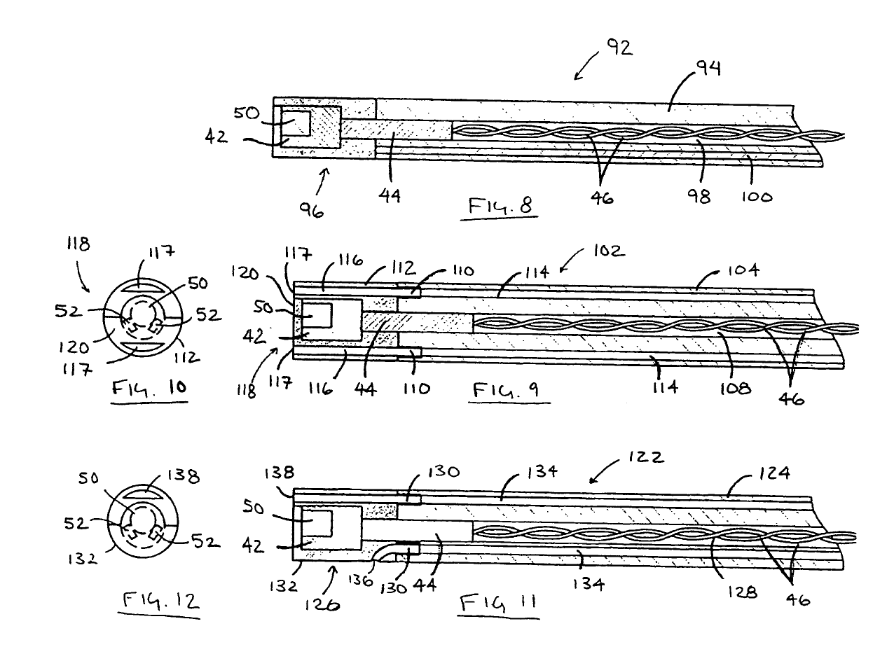

Referring to FIG. 4, an exemplary embodiment of camera assembly 16 is shown without housing 48. As shown, camera head 42 includes optic 50 and is coupled to flex circuit 44 by a plurality of conductors 45. It should be appreciated that conductors 45 may be wires or conductors 45 may be configured as a portion of the flex circuit either as wires in a flexible ribbon or printed conductors forming a flexible ribbon. Flex circuit 44 includes an optional stiffener 54, such as a flexible printed circuit board substrate, which provides a stiffened surface for mounting circuitry components 56, if desired. Any known material may be used, such as Mylar. Stiffener 54 is selected so that it does not impede the flexibility of shaft 12 near second end 20.

Flex circuit 44 may include circuitry that is configured to support any optical features of camera assembly 16. For example, in some embodiments, the flex circuit may be configured to provide voltage regulation from 5V to 3.3V or more, generally a higher voltage to the image sensor's required voltage. Alternatively or additionally, flex circuit 44 may filter electrical noise in the power supply delivered to it from the proximal end of the device. Filtering power supply noise close to the image sensor results in lower image noise in the captured image, which translates into higher quality images. Alternatively or additionally, flex circuit 44 may be configured to connect a wire to the image sensor chip itself. On the output side, it may filter the output video signal such that high frequencies which cause EMI (Electromagnetic Interference) are dampened at the source and do not leak into the outside environment. Preferably, flex circuit 44 is potted with a flexible, electrically insulative potting material so that circuitry components 56 are structurally supported on stiffener 54 and so that flex circuit 44 is structurally coupled to camera head 42. The potting material may be any potting material known in the art that provides the desired flexibility and insulative qualities.

Wires 46 are electrically coupled to and extend from flex circuit 44. A portion of flex circuit 44 and wires 46 extend into lumen 32 of shaft 12 toward handle 14.

In the present embodiment, distal camera housing assembly 48 is constructed from two housing members 48a and 48b and encloses, or partially encloses, camera head 42 and a portion of flex circuit 44 adjacent camera head 42, as shown in FIGS. 5 and 6. Housing members 48a and 48b are generally half-cylinder members that combine to form a generally cylindrical distal housing assembly 48. Each member 48a and 48b includes cavity 58 and channel 60. Cavities 58 are configured so that when the two members 48a and 48b are combined, cavities 58 combine to house camera head 42 and channels 60 combine to form an aperture through housing that allows the potted flex circuit 44 to extend from camera head 42 out of housing 48 and into lumen 32 of shaft 12.

Each of housing members 48a and 48b may include a fluid port 40. Each fluid port 40 is configured to be received by an end portion of a respective fluid conduit 34 included in shaft 12 so that fluid conduit 34 may be extended to lateral fluid inlets/outlets 62 of housing members 48a and 48b. It should be appreciated that filters or other screen members may be provided at the fluid conduit inlets/outlets to prevent the conduits from becoming clogged.

In the present embodiment, housing member 48b also includes an optional optical face 64. Optical face 64 extends over optic 50 and light sources 52, but is optically clear so that images of the target tissue and illumination provided by light sources 52 may transmit through optical face 64. It will be appreciated that, when included, optical face 64 preferably is provided entirely on one or the other of housing members 48a and 48b so that there is no interface between housing members 48a, 48b that extends across either optic 50 or light sources 52. It will be appreciated that optical face 64 may be any shape that provides an optically accurate image and may be constructed to provide magnification if desired. For example, optical face may be planar or curved, and if curved, it may be spherical or aspheric. It should be appreciated, however, that camera head 42 may be constructed so that it is fully or partially waterproof so that optical face 64 need not be employed. For example, the front surface of camera head 42 may be waterproof and exposed and housing members 48a and 48b may combine to form a generally tubular camera housing 48.

Referring to FIG. 7, another embodiment of the shaft of an optical device of the present invention will be described. Shaft 72 includes elongate body member 74 that is configured to extend between a camera assembly 76 and handle (not shown). It will be appreciated that the components of camera assembly 76 that are substantially identical to the components of camera assembly 16, described above, will be referred to by identical reference numbers and will not be further described.

As in the previously described embodiment, body member 74 defines lumen 78 that is configured to receive a portion of flex circuit 44 and a plurality of wires 46 extending from flex circuit 44 of camera assembly 76 toward a handle. Shaft 72 also provides a single fluid conduit 80 that extends generally longitudinally through shaft 72 and is fluidly coupled to port 86, which forms a portion of fluid conduit 84 of camera housing 82. Similar to the previously described embodiment, fluid conduit 84 of camera housing 82 terminates at lateral inlet/outlet 88.

Referring to FIG. 8, in another embodiment, shaft 92 includes elongate body member 94 that defines lumen 98 configured to receive a portion of flex circuit 44 and a plurality of wires 46 extending from flex circuit 44 of camera assembly 96 toward a handle (not shown), but body member 94 does not provide any fluid conduits. Shaft 92 includes a shaping member 100 that provides shaft 92 with shapeability. Shaping member 100 may be a bendable or articulated wire that is embedded within shaft 92 or inserted into a lumen of shaft 92. Shaping member 100 may be constructed from any bendable material such as plastic or metal.

Referring to FIGS. 9 and 10, in yet another embodiment shaft 102 is constructed from elongate body member 104 that defines lumen 108 and a pair of fluid conduits 114. Lumen 108 extends longitudinally through body member 104 and is configured to receive a portion of flex circuit 44 and a plurality of wires 46 extending from flex circuit 44 of camera assembly 106 toward a handle (not shown). Fluid conduits 114 also extend longitudinally through body member 104 and are fluidly coupled to fluid ports 110 of camera housing 112. Fluid conduits 116 extend through camera housing 112 between ports 110 and inlets/outlets 117 that are disposed on a tip 118 of camera assembly 106 adjacent optical face 120. Although inlets/outlets 117 are illustrated as semi-circular it will be appreciated that inlets/outlets 117 may be provided in any desired shape.

Referring to FIGS. 11 and 12, shaft 122 will be described. Shaft 122 generally includes body member 124 that defines lumen 128 and fluid conduits 134 that extend longitudinally through body member 124. Lumen 128 is configured to receive a portion of flex circuit 44 and a plurality of wires 46 extending from flex circuit 44 of camera assembly 126 toward a handle (not shown). Fluid conduits 134 are fluidly coupled to ports 130 of camera housing 132. In the present embodiment, one fluid conduit 134 extends to a lateral inlet/outlet 136 and the other fluid conduit 134 extends to a tip inlet/outlet 138.

Referring to FIGS. 13 and 14, shaft 142 will be described. Shaft 142 includes body member 144 that has a larger diameter than that of the previous embodiments so that an additional access lumen 150, or working channel, may be provided. Similar to the previous embodiments, body member 144 defines lumen 153 and fluid conduits 154 that extend generally longitudinally through body member 144. Similar to the previously described embodiments, fluid conduits 154 are fluidly coupled to ports 149 and fluid conduits 147 that terminate in inlets/outlets 151 of camera housing 148.

Access lumen 150 also extends through body member 144 generally longitudinally and access lumen 152 extends through camera housing 148 and aligns with access lumen 150 so that a single continuous access lumen is provided through body member 144 and camera assembly 146. Access lumens 150 and 152 may be used as working channels and sized so that surgical devices may be advanced through the lumens to a target location. For example, lumens 150 and 152 may be sized to receive a curette, scissors, a cytological brush or another biopsy tool.

A sealing member 156 is provided adjacent an outlet 158 of access lumen 152 to provide a fluid seal while allowing a device to be advanced therethrough. For example, sealing member 156 may be a membrane that includes a slit that may be penetrated by a surgical instrument. As a further example, sealing member 156 may be constructed from overlapping flexible petals that may be flexed by a surgical instrument to provide an aperture. As a still further example, sealing member 156 may be an annular membrane that is folded to seal outlet 158 but may be unfolded to permit passage of a surgical device.

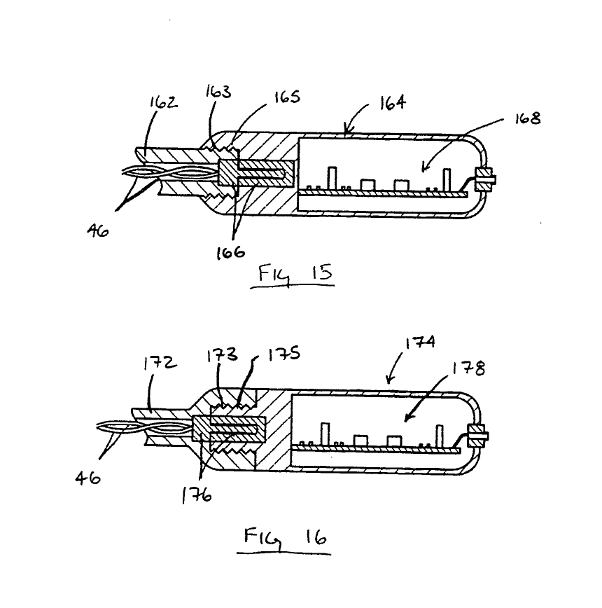

Referring to FIGS. 15-17, various embodiments of a removable interface between a shaft and a handle of an optical device will be described. In all of the embodiments, the interface is configured so that the handle may be easily removed from the shaft so that the components may be separated and discarded, cleaned and/or sterilized. Preferably, the shaft portion of the optical device is constructed so that it is disposable and supplied in a sterilized state and the handle portion is protected so that no sterilization is required. In the embodiment shown in FIG. 15, shaft 162 includes a threaded outer surface 163 that is configured to mate with a threaded inner surface 165 of handle 164. A connector 166, may be provided to electrically couple a camera assembly (not shown) to circuitry 168 housed in handle 164. Connector 166 may be any connector that provides a sufficient number of conductors to electrically couple the components. For example, connector 166 may be a headphone type connector, such as a 2.5 mm or 3.5 mm headphone jack.

In a further embodiment, shown in FIG. 16, shaft 172 includes a threaded inner surface 173 that is configured to mate with a threaded outer surface 175 of handle 174. Coupling the threaded surfaces of shaft 172 and handle 174 assures that connector 176 is engaged so that circuitry of a camera assembly is electrically coupled to circuitry 178 housed in handle 174. In such an embodiment, the portion of shaft 172 adjacent handle 174 has a diameter that generally matches the diameter of handle 174. As a result, it is less likely that a portion of handle 174 will come into contact with body fluids or tissues during use.

In a still further embodiment, shown in FIG. 17, sleeve 186 is rotatably coupled to shaft 182. Sleeve 186 includes a threaded inner surface 187 that is configured to mate with a threaded outer surface 185 of handle 184. An advantage of such a configuration is that during coupling, shaft 182 and handle 184 are not required to rotate relative to each other. As a result, a greater selection of electrical connectors may be utilized to electrically couple the camera assembly housed in shaft 182 and circuitry 188 and software housed in handle 184.

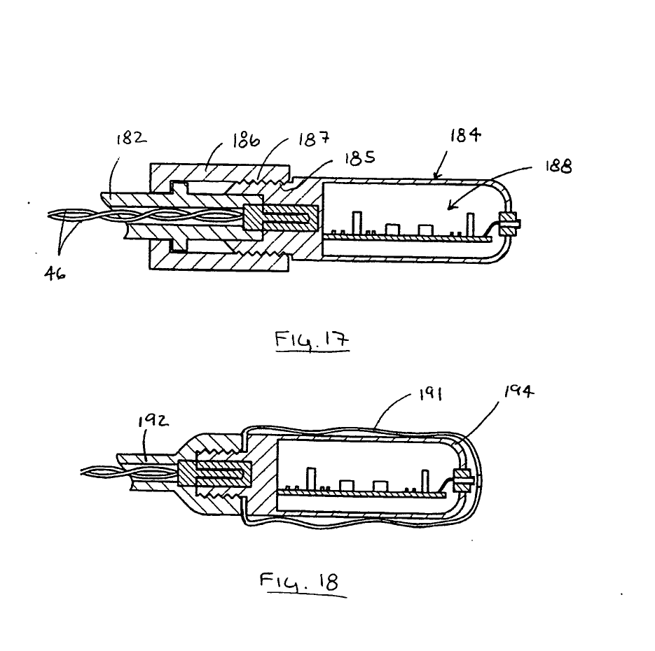

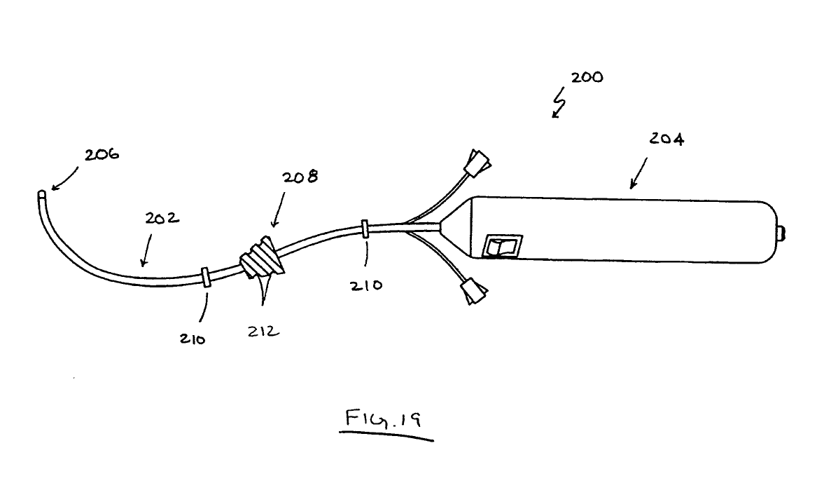

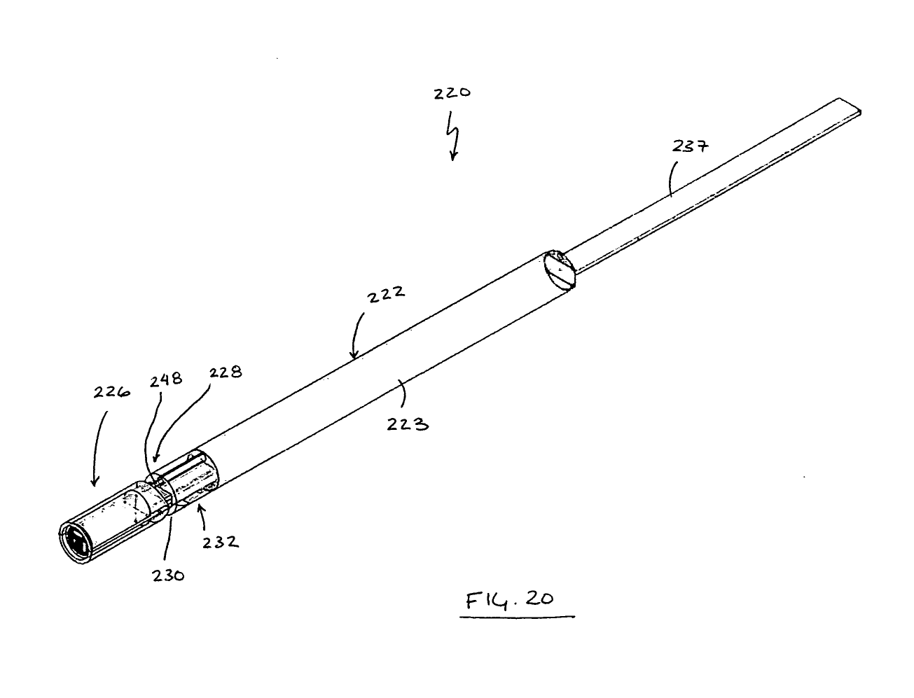

Referring to FIG. 18, protective sleeve 191 may envelope handle 194 to prevent bodily fluids or tissue from contacting handle 194. Protective sleeve 191 is generally a bag that includes at least one opening so that shaft 192 and handle 194 may be coupled as described above. The portion of protective sleeve 191 surrounding the opening is retained between shaft 192 and handle 194 when they are coupled so that handle 194 is completely enclosed. It should be appreciated that additional openings may be provided to provide access to an output connector. Any material may be used to construct protective sleeve 191, such as latex or vinyl, that prevents passage of bodily fluids. It should be appreciated that the shaft may also include an integral sleeve that covers the handle and protects it from exposure to fluids and/or tissues. For example, sleeve 191 may be permanently attached to shaft 192 and may include an opening so that handle 194 may be inserted.

It will be appreciated that any combination of structural and electrical connection may be provided. For example, rather than including a threaded connection between the shaft and handle, a “submarine-hatch” or bayonet type connection may be provided. Such connection provides a connection with fewer relative rotations and includes a fixed relative orientation between the components when fully engaged. Also the electrical connection between the shaft and the handle may be by mating pigtails so that some relative rotation between the shaft and the handle may be accommodated.

As a further example, a friction fit may be provided between the handle and shaft. For example, the shaft may include an engagement portion that has an inner diameter that is slightly larger than an outer diameter of an engagement portion of the handle and the engagement portion of the shaft may be slid over the engagement portion of the handle. Additionally, sealing members, such as o-rings may be disposed between the engagement portions of the shaft and handle to increase the frictional interface and/or to provide a liquid barrier.

Referring to FIG. 19, optical device 200 will be described. Optical device 200 generally includes a flexible shaft 202, handle 204, camera assembly 206 and an optional applicator member 208. Similar to the previously described embodiments, shaft 202 extends between handle 204 and camera assembly 206 and is generally shapeable. Applicator member 208 is slidably coupled to shaft 202 between first and second stops 210 that limit the sliding travel of applicator member 208 on shaft 202. However, it should be appreciated that applicator member 208, if included, may be fixedly coupled to shaft 202 or applicator member may be slidably coupled and stops 210 may be omitted.

Applicator member 208 may be included to seal, or plug, a body orifice that is being inspected with optical device 200. Such a seal may be desired, for example, during examination of a uterus so that it may be distended with saline, CO2 or another fluid. Applicator member 208 may be constructed from any pliable material sufficient to fluidly seal a body orifice. For example, applicator member 208 may have a conical or frustoconical shape, and be constructed from foam or silicone and it may have surface features such as a helical groove 212 that improves the fluid seal, or have a smooth lateral surface.

A coupling feature also may be provided on shaft 202 so that applicator member 208 may be temporarily coupled to shaft 202. Such a temporary coupling may be desired so that applicator member 208 may be precisely placed in a body orifice. The coupling may be particularly advantageous when applicator member 208 includes a helical groove so that applicator member 208 may be rotated into place.

In a further embodiment, an outer tube may be included around shaft 202. The outer tube may be coupled to applicator 208 so that sliding the outer tube relative to shaft 202 moves applicator 208 longitudinally along shaft 202. Preferably, the outer tube is configured so that it may be bunched upon shaft 202 or extended out unwrinkled upon shaft 202. For example, the outer tube may include a crunchable portion. The crunchable portion may be a bellows that is self-locking in a compressed, partially compressed or extended configuration so that the outer tube may be moved relative to shaft 202.

It should further be appreciated that applicator 208 may be replaced by or used in addition to an inflatable balloon. The inflatable balloon may be coupled to an outer surface of shaft 202 such as at a distal end and an inflation lumen may be provided through shaft so that inflation fluid, such as saline or CO2 may be used to inflate the balloon to seal, or plug, an orifice or to distend a cavity.

As described above, circuitry and software for the optical device may be included as part of the camera assembly and/or in the handle. Preferably the circuitry and software is provided in the handle so that it may be reused with disposable shafts. The circuitry includes a controller that preferably includes embedded software that provides the user with the ability to control the camera assembly and to manipulate the image data gathered by the camera assembly. For example, the circuitry may provide control over orientation of the camera head, focus and/or zoom. The circuitry may also provide control over brightness and/or color of the light provided by the illumination source. The circuitry may also include universal serial bus (USB) components so the optical device of the present invention may be easily coupled to peripheral devices, for example, to a personal computer, and may also include a connection to a national television standards committee (NTSC) monitor via a standard recording corporation of America (RCA) cable or any other cable. Moreover, the circuitry may include wireless components to provide wireless communication between the optical device and peripheral devices (e.g., monitors and/or recording devices). Additionally, the circuitry may include components so that the output connector shown in FIG. 1 may be directly connected to a monitor or other video screen or to a recording device. The circuitry provided in the handle may also be capable of storing still images, video and/or audio.

In an embodiment, a small display screen is integrated into the handle of the device. In another embodiment, a small display device is plugged into a connector provided on the handle of the device so that the display is supported by the handle.

Power may be provided by a battery or an external power source electrically coupled to the handle circuitry. In embodiments utilizing a battery, the battery may be rechargeable if desired and circuitry may be provided so that the battery may be charged while it is in the handle. For example, a power adapter may be provided that plugs directly into the circuitry. Alternatively, inductive charging circuitry may be included in the handle and a complimentary cradle provided so that inserting the handle into the cradle will inductively charge the battery.

A clip member may also be provided on the handle of the optical device so that the optical device may be temporarily clipped to a support device. For example, a support device may be temporarily coupled (e.g., by an adhesive strip) to a patient so that the optical device may be held in position without requiring the user to hold it. The clip member may also be configured so that the optical device can be coupled to another surgical device during a procedure, such as a speculum.

Additionally, mechanical control may be provided on the handle so that the tip of the shaft may be flexed, pointed, rotated and/or translated during use. For example, a slide switch may be included on the handle that is linked to the end of the shaft adjacent the camera assembly or directly to the camera assembly. The link may be configured so that manipulation of the slide switch causes the shaft to be flexed and the camera assembly and camera head to be rotated relative to the shaft or causes the camera to move, such as by bending or flexing, relative to the shaft. It should be appreciated that the camera head may be rotated or hinged over a large range of motion, such as 0-200 degrees in either direction from a zero angle position. Preferably, the control is configured so that the camera head may be rotated over a range of +/−0-30 degrees. In embodiments utilizing a head that rotates relative to the shaft about an axis generally parallel to the longitudinal axis of the shaft the camera head, as described in greater detail below, may be rotated over a range of 0-200 degrees, but preferably between 160-180 degrees. It will be appreciated that the shapeability of the shaft also allows a user to preset the orientation of the camera head as desired.

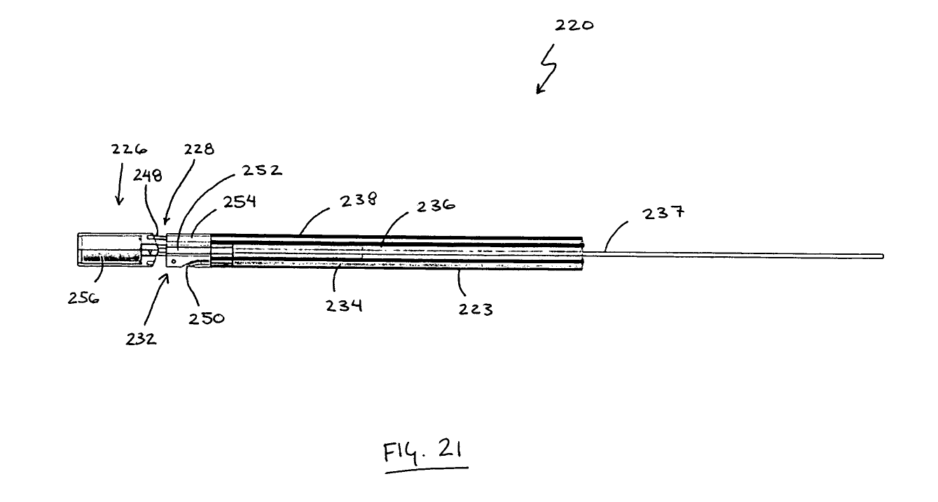

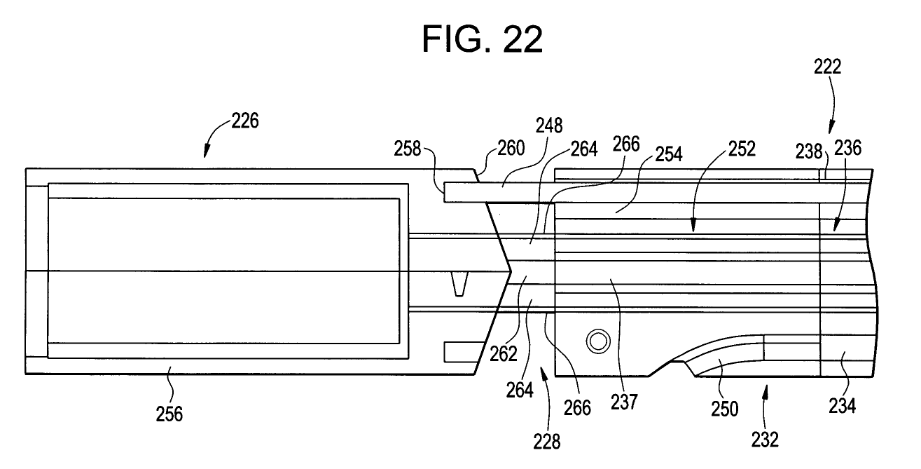

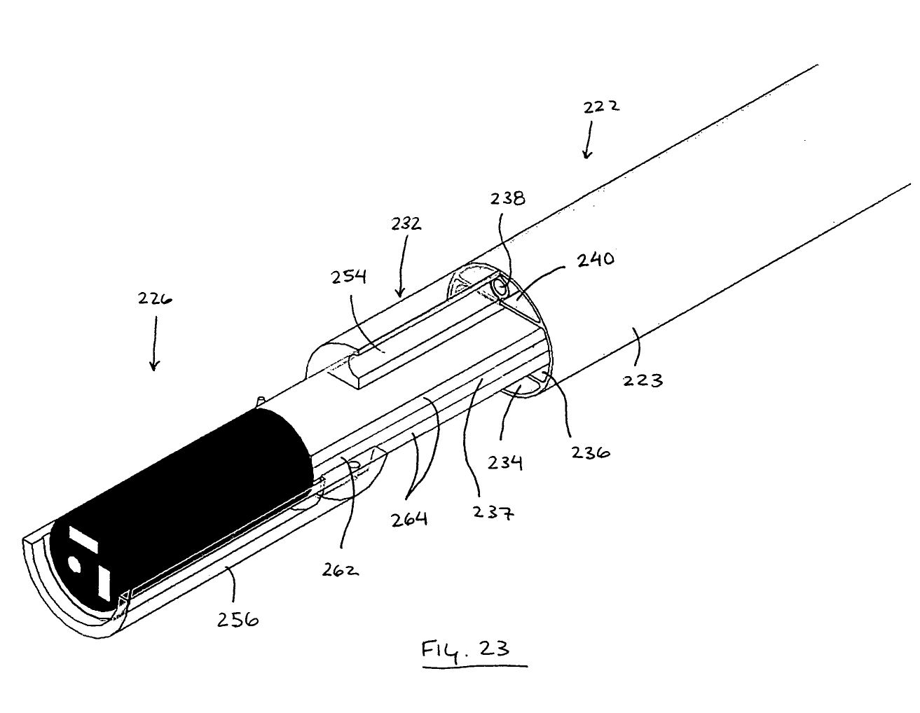

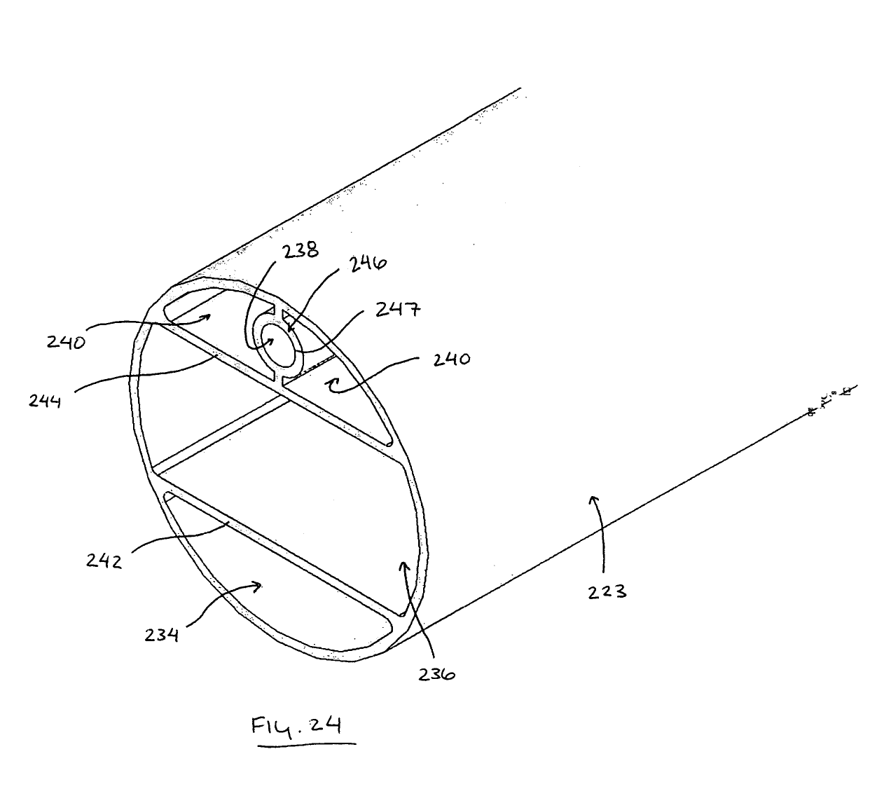

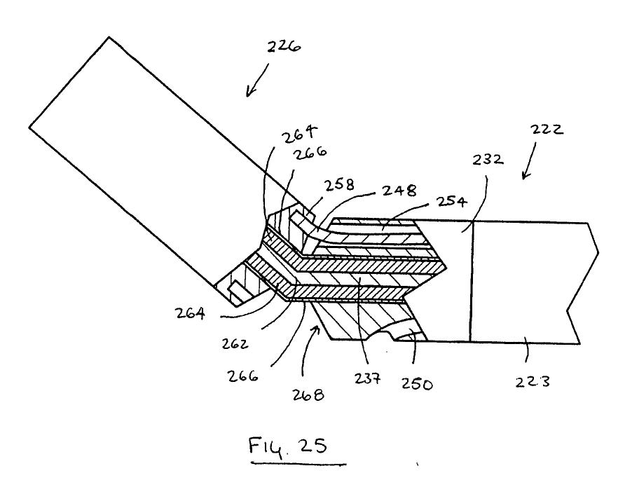

Referring to FIGS. 20-24 an embodiment of the optical device will be described that includes camera assembly 226 that is coupled to a shaft 222 by a hinge member 228 so that camera assembly 226 may be rotated relative to shaft 222 along an axis that is perpendicular to shaft 222. Similar to the previously described embodiments, shaft 222 includes elongate body member 223 and shaft tip 232 that extends between a handle (not shown) and camera assembly 226. Elongate body member 223 is generally flexible and is preferably shapeable.

Elongate body member 223 includes a first, proximal, end that is coupled to the handle and a second, distal, end 230 that is coupled to an optional shaft tip 232. Elongate body member 223 defines a plurality of lumens, as shown in FIG. 24. In particular, elongate body member 223 includes fluid conduit 234, lumen 236 for receiving flexible printed circuit, e.g., flex circuit 237, and wires, pull wire lumen 238 and stiffening wire lumens 240. Elongate body 223 preferably is extruded.

Fluid conduit 234 extends longitudinally through elongate body member 223 and provides introduction and/or aspiration of saline, CO2 or any other fluid into or out of a target site. Fluid conduit 234 is defined by a first wall 242 that forms a chord extending across the interior of elongate body member 223 and the outer wall of elongate body member 223.

Lumen 236 is defined by first wall 242, second wall 244 and the outer wall of elongate body member 223. Second wall 244 is parallel and spaced from first wall 242 and also forms a chord extending across the interior of elongate body 223. Lumen 236 of the present embodiment has a generally rectangular cross-sectional shape. The size and shape of lumen 236 is selected to receive flexible printed circuit 237 while reducing the overall cross-sectional area of elongate body member 223.

Pull wire lumen 238 and stiffening wire lumens 240 are formed between second wall 244 and the outer wall of elongate body member 223. In particular, pull wire lumen 238 is defined by a third wall 246 suspended between second wall 244 and the outer wall of elongate body member 223 that includes cylindrical tubular portion 247. Tubular portion 247 extends longitudinally through elongate body member 223. Pull wire lumen 238 is configured to receive a pull wire 248, shown in FIGS. 20-22, that extends between the handle and camera assembly 226 so that hinge member 228 may be actuated (i.e., so that camera assembly 226 may be rotated relative to shaft 222).

Stiffening wire lumens 240 are provided on either side of third wall 246 adjacent pull wire lumen 238. Stiffening wire lumens 240 may be used to receive stiffening wires so that the shape of the flexible elongate body member 223 may be manipulated. For example, a stiffening wire having a bent configuration may be inserted into stiffening wire lumen 240 so that elongate body member 223 is urged to take the shape of the stiffening wire. Alternatively, a stiffening wire having variable stiffness over its length may be inserted into stiffening wire lumen 240 to impart the same variable stiffness to elongate body member 223.

Optional shaft tip 232 is coupled to the distal second end 230 of shaft 222. Shaft tip 232 preferably is permanently coupled to shaft 222. It should be appreciated that shaft tip 232 may be assembled from generally semi-cylindrical members that interlock to form the generally cylindrical shaft tip assembly 232, if desired.

Shaft tip 232 includes lumens that communicate with some or all of the lumens of elongate body 223. For example, shaft tip 232 defines fluid conduit 250, lumen 252 for flex circuit 237 and pull wire lumen 254, but does not include lumens in communication with stiffening wire lumens 240. However, it should be appreciated that shaft tip 232 may include stiffening wire lumens if desired.

Fluid conduit 250 extends through a portion of shaft tip 232 and exits a side wall of shaft tip 232. It should be appreciated that fluid conduit 250 may alternatively extend longitudinally through shaft tip 232 so that it exits along a distal face of the tip, if desired.

Lumen 252 extends longitudinally through shaft tip 232 generally along the central longitudinal axis of shaft 222. The size and shape of lumen 252 are selected to receive flexible printed circuit 237 and wires extending between flexible printed circuit 237 and the distal end of shaft 222.

Pull wire lumen 254 extends longitudinally through shaft tip 232 and provides a pathway for a pull wire to extend through shaft 222 and into camera assembly 226. Pull wire lumen 254 is sized and oriented so that pull wire 248 is slidable within shaft 222 and aligned with a pull wire mounting feature 258 included on camera assembly 226. In the present embodiment, pull wire mounting feature 258 is a blind hole that extends into camera assembly 226 from chamfered surface 260 and a distal end of pull wire 248 is mechanically bonded in the blind hole. For example, the distal tip of pull wire 248 may be glued in the hole.

In embodiments utilizing a shaft tip assembled from a plurality of tip members, any of the lumens may be defined by the combination of the tip members. For example, each of tip members may include a generally U-shaped or semi-cylindrical channel that defines a portion of lumen 252. When the tip members are assembled into shaft tip 232 the channels align to form lumen 252. It should further be appreciated that shaft tip 232 may be omitted if desired. For example, a distal end of the elongate body member may be shrink-wrapped or otherwise sealed without employing a separate shaft tip. In such an embodiment, the fluid conduits extending through the elongate body member may include an exit at, or adjacent, the distal end of the elongate body member.

Camera assembly 226 is coupled to shaft 222 via hinge member 228 so that camera assembly 226 may be flexed, or rotated, relative to shaft 222. The structure of camera assembly is substantially identical to the camera assemblies described above. However, camera housing 256 has been modified to provide for additional relative motion between camera assembly 226 and shaft 222. In particular, the proximal end of camera housing 256 (i.e., the end of camera housing nearest shaft 222) includes chamfered surface 260 so that the available relative motion between camera assembly 226 and shaft 222 is increased.

Hinge member 228 is constructed from a hinge portion 262 of flex circuit 237. Hinge portion 262 of flex circuit 237 extends between shaft 222 and camera assembly 226 and permits relative motion between shaft 222 and camera assembly 226. A pair of protective sheets 264 sandwich hinge portion 262. In the present embodiment, protective sheets 264 are sandwiched with flex circuit 237, but they are not attached directly to each other.

Protective sheets 264 and hinge portion 262 may be enclosed in waterproof shrink-wrap 266, as shown. The ends of shrink wrap 266 extend into shaft 222 and camera assembly 226 and are coupled therein to provide a liquid tight seal. The liquid tight seal between shrink-wrap 266 and each of shaft 222 and camera assembly 226 assure that flex circuit 237 is protected from ingress of fluids.

Pull wire 248 provides user control over the bending of hinge member 228. In the present embodiment, hinge member 228 is biased to bend along a single axis due to the shape of flex circuit 237. In particular, hinge portion 262 of flex circuit 237 has a generally rectangular cross-section with width that exceeds the height. As a result, hinge member 228 is biased to bend along an axis that is parallel to the width dimension of flex circuit 237. Because hinge member 228 is biased to bend along a single axis, a single pull wire 248 may be employed. Pull wire 248 is configured so that when a user applies tension to pull wire 248 it pulls one side of camera assembly 226 toward shaft 222 thereby bending hinge member 228.

Additionally, hinge member 228 may be constructed so that it is biased to a particular position within the range of motion. For example, hinge member 228 may be biased so that there is no bend in hinge member 228 and the optical axis of camera assembly 226 is generally aligned with the longitudinal axis of shaft 222 (referred to herein as a “zero angle position”). Alternatively, hinge member 228 may be biased to a bent position. The tension of pull wire 248 may be adjustable so that camera assembly is set to any position within the range of motion. For example, the bias of hinge member 228 may be configured so that hinge member 228 is naturally bent to a first rotated position. The tension of pull wire 248 may be adjusted, or initially set, so that hinge member is returned to the zero angle position. The hinge member 228 may then be bent to a second rotated position by further increasing tension on pull wire. As a result, a single pull wire 248 may be used and maintained in constant tension while providing a camera assembly 226 that is capable of bending to positions on either side of the zero angle position.

Pull wire 248 may also be constructed so that it is semi-rigid so that both tension and compression are transmitted to camera assembly 226. In such an embodiment, hinge member 228 may be biased to the zero angle position, then pushing pull wire 248 places camera assembly 226 in a first rotated position and pulling pull wire 248 places camera assembly 226 in a second rotated position and the first and second rotated positions correspond to opposite directions of rotation.

Referring to FIG. 25, shaft tip 232 may include a chamfered distal surface 268. Chamfered distal surface 268 provides for a greater angle of rotation of camera assembly 226 relative to shaft 222. In particular, interference between camera assembly 226 and shaft 222 is avoided over a greater angular travel of camera assembly 226 relative to shaft 222. It should be appreciated that chamfered surfaces 260 and 268 may have any chamfer angle desired.

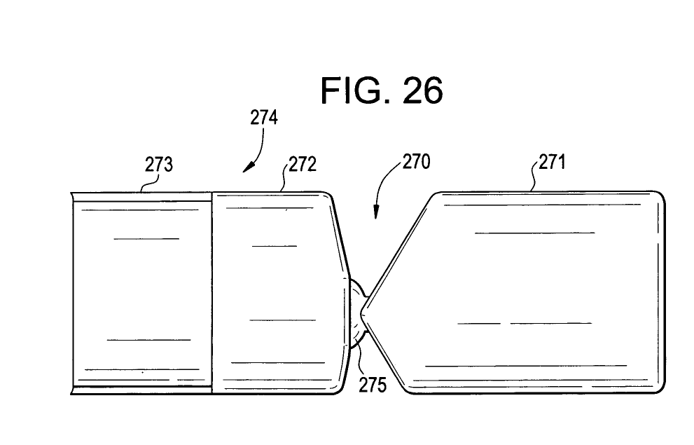

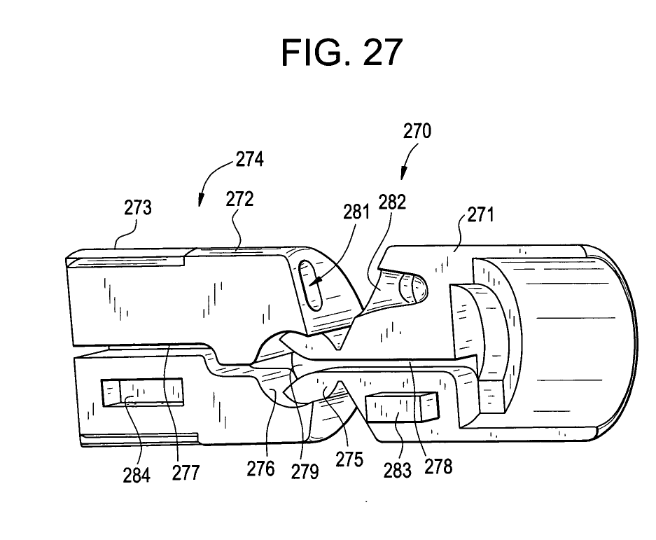

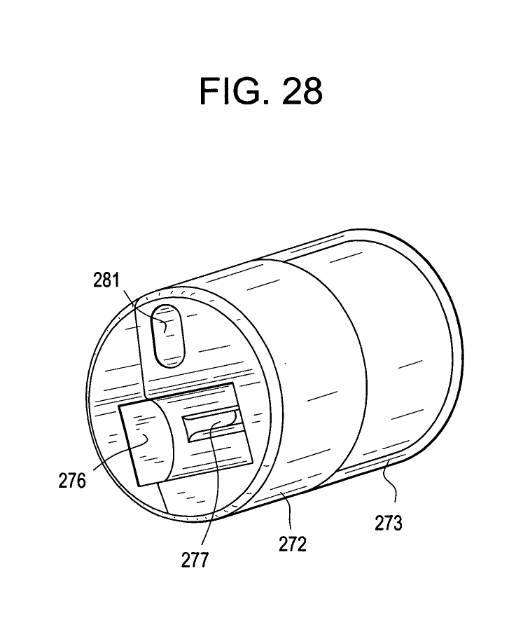

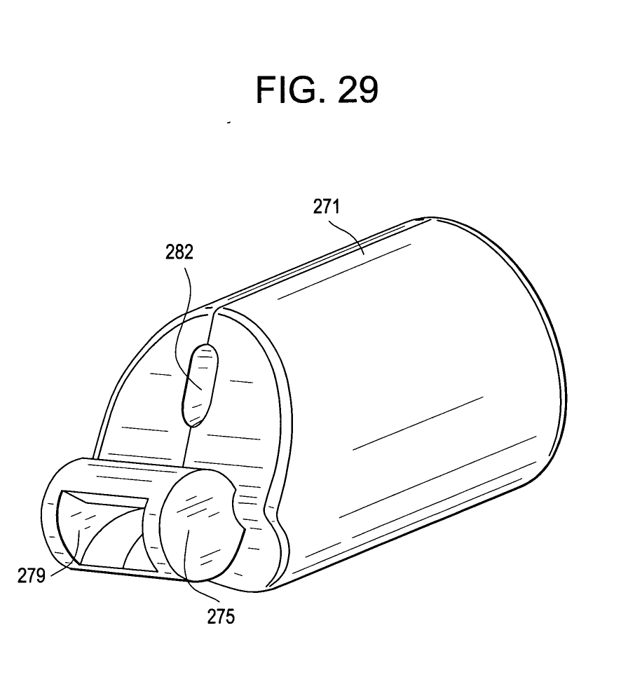

Referring to FIGS. 26-29 a distal end of another embodiment of the optical device will be described that includes another embodiment of a hinge member between the shaft and the camera assembly. In particular, hinge member 270 couples camera housing 271 of a camera assembly to shaft tip 272 so that the camera assembly may be selectively rotated relative to shaft 274 of the optical device. Similar to the previously described embodiments, shaft 274 includes elongate body member 273 and shaft tip 272 that extends between a handle (not shown) and camera housing 271 of a camera assembly. Elongate body member 273 is generally flexible and is preferably shapeable.

Hinge member 270 movably couples shaft tip 272 with camera housing 271 and is generally formed from cylindrical projection 275 that extends proximally from a proximal end of camera housing 271 and a cylindrical pocket 276 included in the distal end of shaft tip 272. In the present embodiment, cylindrical projection 275 and pocket 276 are oriented so that each has a longitudinal axis that is perpendicular to a longitudinal axis of shaft 274. As a result, when cylindrical projection 275 is received in cylindrical pocket 276 they form a movable joint that allows camera housing 271 to rotate relative to shaft 274 along an axis that is also perpendicular to the longitudinal axis of shaft 274.

Similar to the previously described embodiments, a flexible printed circuit may be provided that spans hinge member 270 so that the camera assembly may be electrically coupled to electronics included in shaft 274 and/or the handle. Shaft tip 272 includes a lumen 277 that intersects and includes an opening into cylindrical pocket 276. Camera housing 271 includes lumen 278 that extends through cylindrical projection 275 and is also open to cylindrical pocket 276. The proximal end of lumen 278 includes a tapered opening 279 so that a gradual bend is imparted to a flexible printed circuit extending through lumens 277 and 278 when hinge member 270 is actuated.

Hinge member 270 may be actuated by a pull wire that extends through pull wire lumen 281 and into camera housing 271. In an embodiment, the pull wire is slidably received in lumen 281 so that it may be selectively advanced and/or retracted relative to shaft 274. A distal end of the pull wire is coupled to camera housing 271 at pull wire mounting feature 282, which may be a blind hole and, as shown, may be nonlinear. The distal end of pull wire lumen 281 and the proximal end of pull wire mounting feature 282 may include tapered openings so that a gradual bend is imparted to the pull wire when hinge member 270 is actuated.

In the present embodiment, the rotational axis of hinge member 270 is perpendicular and spaced from the longitudinal axis of shaft tip 272 and camera housing 271. The location of rotational axis of hinge member 270 may be located in any desired position to provide a desired amount of rotation or clearance for channels, such as one or more fluid conduit and/or one or more working channels extending through shaft 274.

Additionally, in the present embodiment, shaft tip 272 and camera housing 271 are each constructed from a plurality of complimentary components. Shaft tip 272 includes components that have a mating interface that intersects cylindrical pocket 276. That configuration simplifies assembly by allowing shaft tip 272 to be assembled over cylindrical projection 275. Furthermore, the multi-piece configuration also allows both shaft tip 272 and camera housing 271 to be assembled over the flexible printed circuit. The components of camera housing 271 and shaft tip 272 may also include alignment features, such as alignment tabs 283 and alignment tab sockets 284, to further simplify assembly.

It should be appreciated that the locations of cylindrical projection 275 and cylindrical pocket 276 may be reversed. For example, cylindrical projection 275 may extend from shaft tip 272 and cylindrical pocket 276 may be included in camera housing 271.

The distal surface of shaft tip 272 and/or the proximal surface of camera housing 271 may also include chamfers to increase the available rotation of hinge member 270. In particular, the chamfers provide clearance so that camera housing 271 does not contact shaft tip 272 at the ends of the rotational travel of hinge member 270.

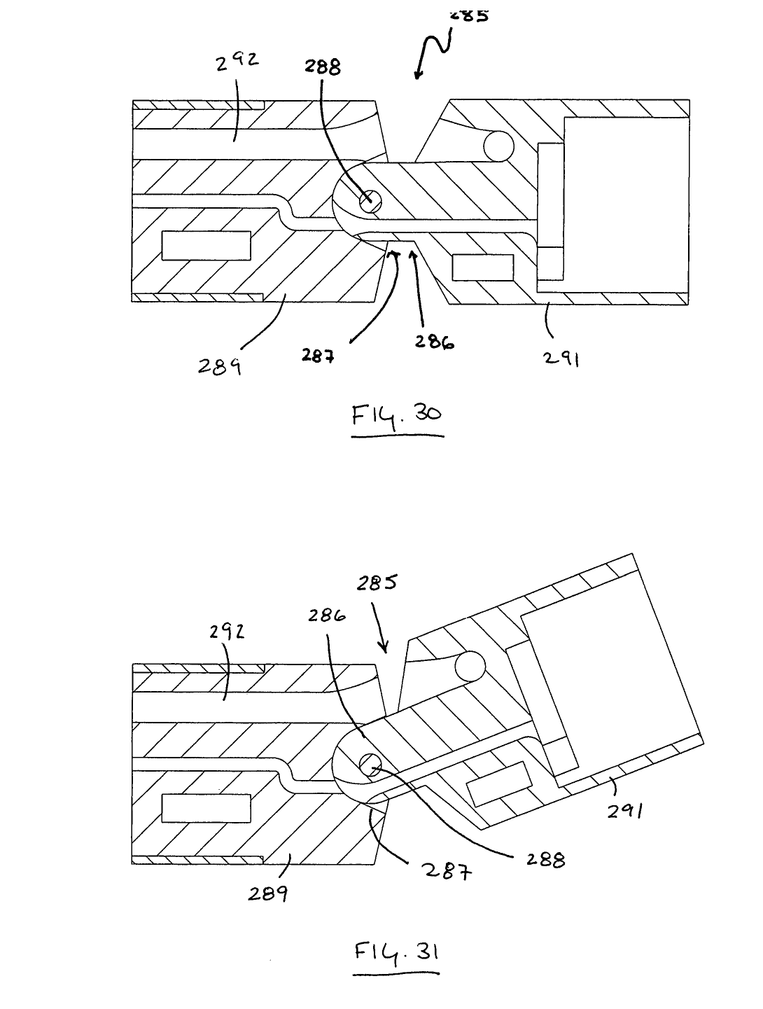

In another embodiment, shown in FIGS. 30 and 31, hinge member 285 includes cylindrical projection 286, cylindrical pocket 287 and hinge axle 288. Cylindrical projection 286 extends into cylindrical pocket 287 and hinge axle 288 extends laterally through cylindrical projection and into sidewalls of shaft tip 289, thereby retaining cylindrical projection 286 within cylindrical pocket 287. Hinge axle 288 may be fixedly coupled to one or the other of cylindrical projection 286 or shaft tip 289 so that cylindrical projection 286 is permitted to rotate within cylindrical pocket 287. Alternatively hinge axle 288 may be freely rotatable with respect to both shaft tip 289 and cylindrical projection 286.

A lumen 290 is included that extends through cylindrical projection 286 and shaft tip 289, across the rotating interface between cylindrical projection 286 and cylindrical pocket 287. Lumen 290 may include tapered portions so that a gradual bend is imparted to a flexible printed circuit extending through lumen 290 when camera housing 291 is rotated relative to shaft tip 289.

In the present embodiment, the axis of rotation of camera housing 291 relative to shaft tip 289 is disposed so that is intersects a central longitudinal axis of shaft tip 289, however, it should be appreciated that the axis of rotation may be located at any radial position relative to the central longitudinal axis of shaft tip 289. Additionally, cylindrical pocket 287 is provided with a tapered opening to allow a desired amount of relative rotation between camera housing 291 and shaft tip 289. The tapered opening of cylindrical pocket 287 allows the cylindrical projection to include parallel sidewalls that extend generally tangentially from the cylindrical surface of cylindrical projection 286. Hinge member 285 is configured to be actuated by a pull wire (not shown) extending through pull wire lumen 292 and coupled to camera housing 291.

The cylindrical hinge configuration provides a hinge member in which the flexible printed circuit remains protected inside the camera housing and shaft through the entire range of travel. Additionally, the configuration obviates the need for including protective sheets or a protective covering, such as shrinkwrap.

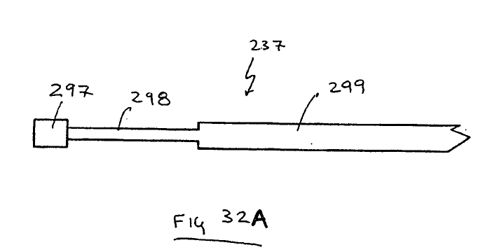

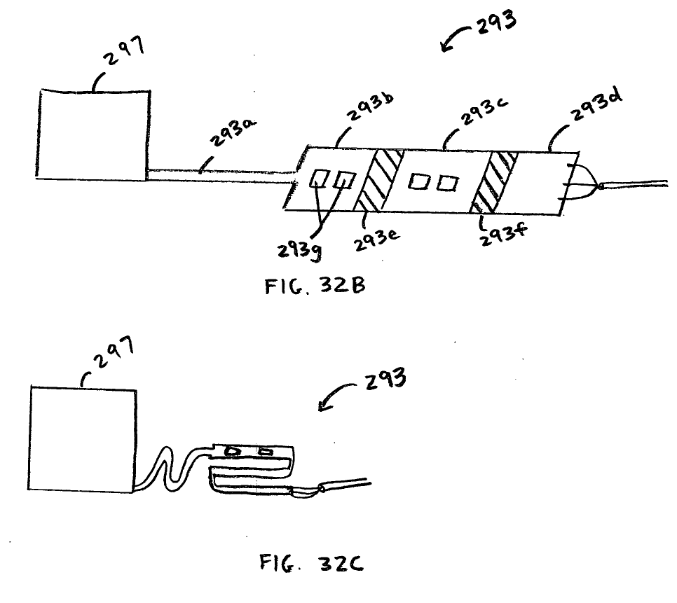

Flexible printed circuit 237 is preferably constructed so that its dimensions vary over its length. For example, as shown in FIG. 32A, flexible printed circuit 237 is coupled to camera head 297 at an end of a first portion 298 of flexible printed circuit 237 that has a smaller transverse dimension than a second portion 299. Hinge portion 262 (e.g., FIG. 25) is included in first portion 298 so that hinge portion has a smaller transverse dimension than the second portion 299. The shape of flexible printed circuit 237 provides for a thin hinge portion 262 while allowing sufficient space for electronic circuits on flexible printed circuit 237.

FIGS. 32B and 32C are perspective views of another embodiment of a flexible printed circuit 293 for inclusion within an optical device in accordance with the present invention. As shown in FIG. 32B, flexible printed circuit 293 includes thinner, flexible portion 293a, and wider, flexible circuit portions 293b, 293c, and 293d separated by fold areas 293e and 293f. One or more of flexible circuit portions 293b, 293c, and 293d (e.g., 293b and 293c) may include circuit component(s) 293g, whereas the other one or more of portions 293b-d (e.g., 293d) may not. In some embodiments, each of flexible circuit portions 293b, 293c, and 293d may have the same length. In some embodiments, flexible printed circuit 293 may be inserted lonigtudinally within the shaft of the optical device. In other embodiments, as shown in FIG. 32C, flexible printed circuit 293 may be folded at the two fold areas by 180 degrees prior to insertion into a device (e.g., a device other than the optical device), thus reducing its length. Thin flexible portion 293a may be folded or otherwise bunched in order to further reduce the length of flexible printed circuit 293.

The optical device of the present invention may also include mechanical control so that the camera assembly may be moved radially outward from the longitudinal axis of the optical device. Such a feature may be used to minimize the dimensions of the device during insertion while providing access to a working channel. As previously described, a working channel may be incorporated to provide a path to a target site through the optical device. In embodiments utilizing a side-by-side configuration of a camera assembly and working channel, the overall outer dimension of the optical device must be increased. However, the device may alternatively include a movable camera assembly that blocks the working channel only during insertion.

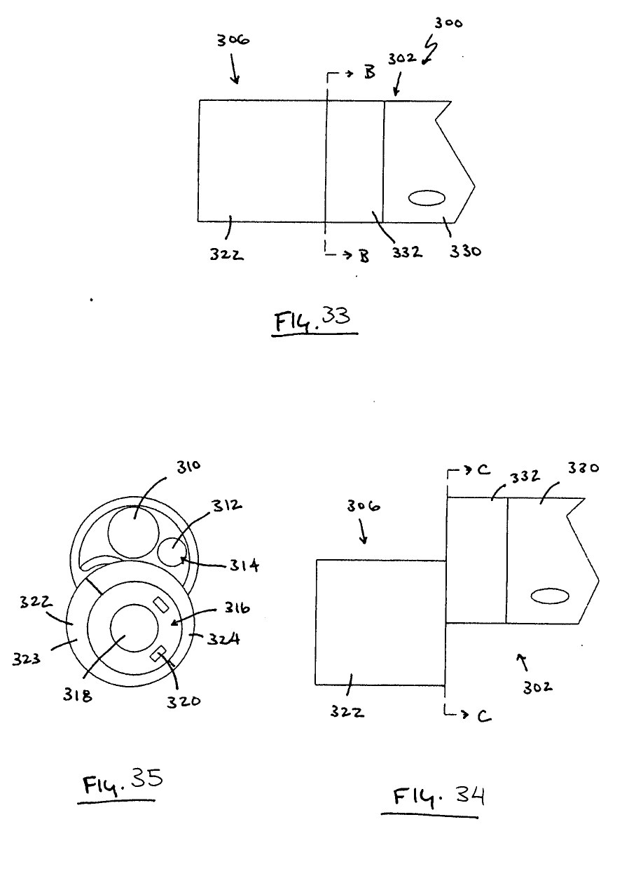

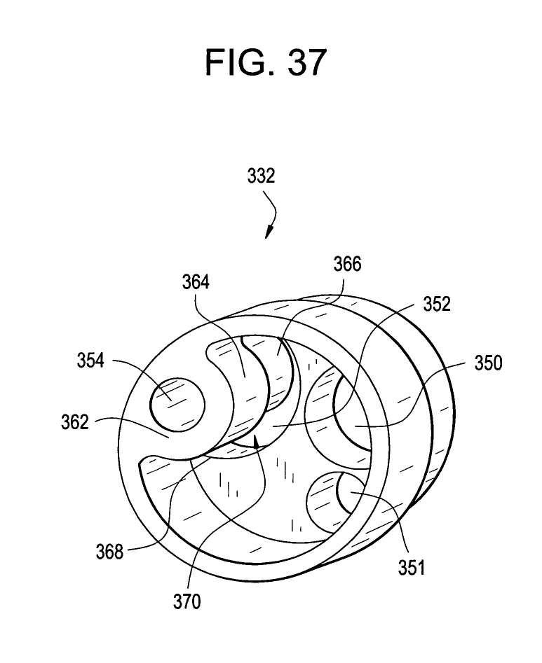

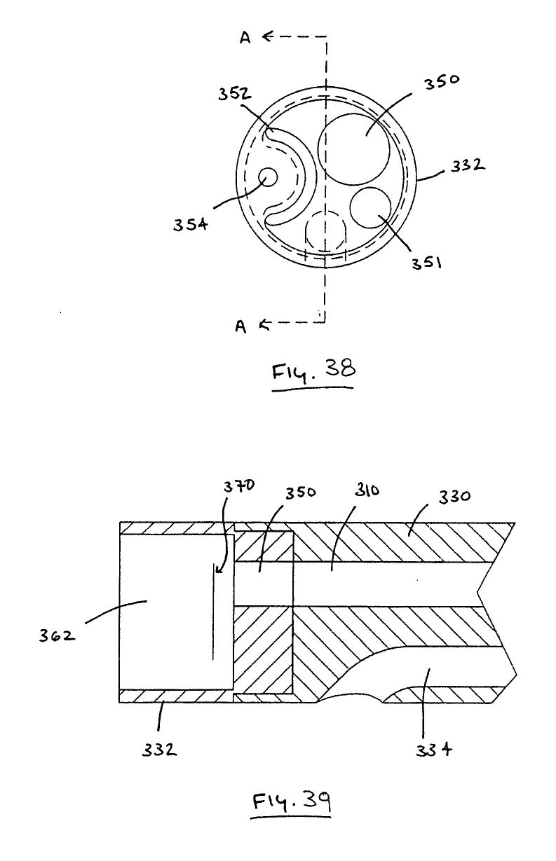

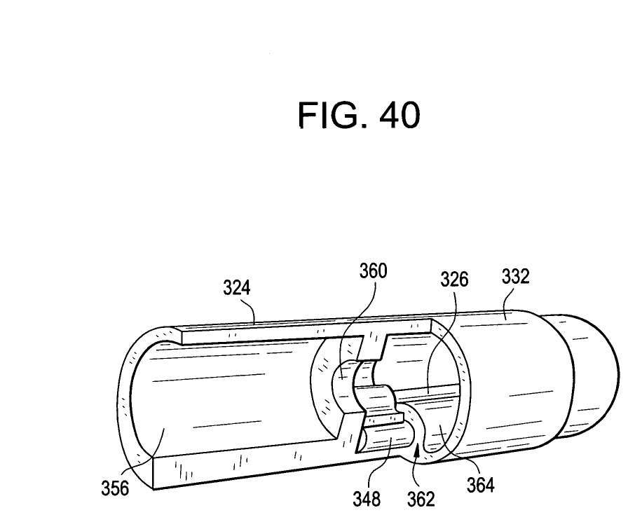

An embodiment of an optical device including a camera assembly that may be moved radially outward will be described with reference to FIGS. 33-42. First referring to FIG. 33, optical device 300 generally includes a shaft 302, a handle (not shown), and camera assembly 306.

Shaft 302 extends between the handle and camera assembly 306 and is flexible and shapeable. Similar to the previously described embodiments, shaft includes elongate body 330 and tip 332 and a first end of shaft 302 is removably coupled to an end of the handle. Shaft 302 is removable to allow disposal of shaft 302 and camera assembly 306 and reuse of the handle.

In the present embodiment, camera assembly 306 is configured so that it may be rotated relative to shaft 302 about an axis that is parallel and spaced from the longitudinal axis of shaft 302. That relative rotation permits optical device 300 to be converted between the first configuration, shown in FIG. 33, and a second configuration in which camera assembly 306 is moved radially outward from the longitudinal axis of shaft 302, as shown in FIGS. 34 and 35.

In the first configuration, the outer dimension of the distal end of optical device 300 is minimized to simplify insertion into a body cavity. In such a configuration, the longitudinal axis of camera assembly 306 is generally coincident with the longitudinal axis of shaft 302.

In the second configuration, camera assembly 306 is rotated relative to shaft 302 so that the longitudinal axis of camera assembly 306 is offset from the longitudinal axis of shaft 302. As shown in FIG. 35, rotation of camera assembly 306 into the second configuration provides clearance for the distal opening 308 of working channel 310 as well as outlet 312 of fluid conduit 314.

Camera assembly 306 includes camera head 316 that is electrically coupled to a processing circuit that is housed in shaft 302 and/or the handle. Camera head 316 includes optic 318 and light sources 320 and is enclosed by camera housing assembly 322.

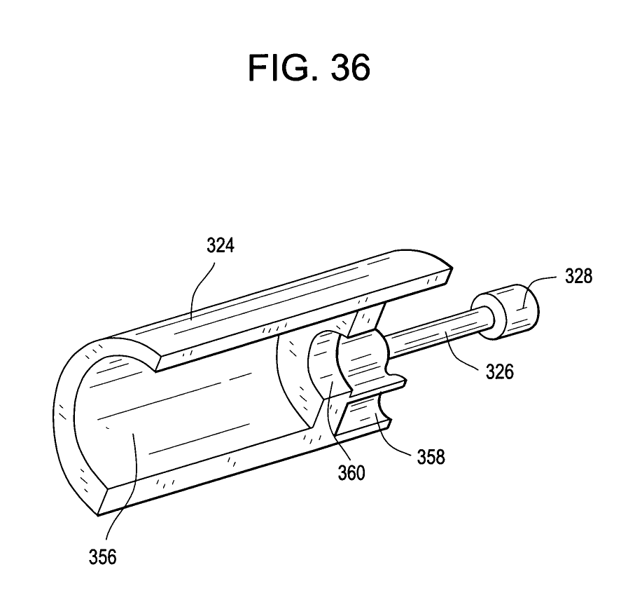

Referring to FIGS. 35 and 36, camera housing assembly 322 includes two housing members 323 and 324 that are coupled together to at least partially enclose camera head 316. In the present embodiment, each of housing members 323, 324 is semi-cylindrical. Housing members 323, 324 combine to define cavity 356 that is configured to receive and at least partially enclose a camera head. Housing members 323, 324 also combine to define aperture 360 that provides a channel for circuitry, such as a flex circuit, to pass between the camera head and shaft 302. Finally, housing members 323, 324 combine to define a hinge pin mounting feature, such as hinge pin aperture 358. As will be discussed in greater detail below, hinge pin aperture 358 receives a distal end of hinge pin 348 and is fixedly coupled thereto. Hinge pin 348 extends through at least a portion of shaft 302 and into camera housing assembly 322 and is rotatable within shaft 302 so that rotation of hinge pin 348 relative to shaft 302 causes housing assembly 322 and an enclosed camera head to rotate relative to shaft 302 thereby placing the optical device into the second, rotated configuration.

Housing member 324 includes an optional locating, and travel limiting, pin 326 that extends from a proximal end of housing member 324. Locating pin 326 is an elongate member that includes a head 328 disposed on the furthest proximal end of locating pin 326. Head 328 is a portion of locating pin 326 that includes an enlarged outer diameter. Head 328 engages a portion of shaft tip assembly 332 to prevent relative translation between shaft 302 and camera assembly 306 in the direction of the longitudinal axis of shaft 302 while allowing camera assembly 306 to rotate relative to shaft 302 over a predetermined rotational travel.

Locating pin 326 and head 328 engage features in shaft tip assembly 332, shown in FIGS. 35 and 37-40. Shaft tip assembly 332 may be constructed from a plurality of shaft tip members if desired or it may be molded as a monolithic body. Tip assembly 332 is fixedly coupled to the distal end of shaft 302. Tip assembly 332 includes at least one fluid conduit 350, hinge pin lumen 354, a working channel 350 (also referred to herein as an “access channel”), and lumen 352 that is configured to receive a portion of a flexible printed circuit or wires, all of which extend through a proximal portion of tip assembly 332 and terminate at a counterbored distal portion of the assembly. Although in the present embodiment, each of the lumens is shown extending only through a proximal portion of tip assembly 332, it should be appreciated that tip assembly need not include the counterbored distal portion and the lumens may extend entirely through tip assembly 332.

Boss 362 is included in the counterbored distal portion of tip assembly 332. Boss 362 defines an outer surface 364 that is generally concentric with hinge pin lumen 354. In the assembled optical device, an outer surface of locating pin 326 included in housing member 324 slides along outer surface 364 during relative motion between camera assembly 306 and shaft 302.

Boss 362 extends from the distal end of tip assembly 332 proximally to a location that is spaced from the distal end of lumen 352. Additionally, lumen 352 is generally crescent or C-shaped and includes a smaller diameter arcuate internal surface 366 that is concentric with hinge pin lumen 354 and a larger diameter arcuate internal surface 368. The radial distance between the longitudinal axis of hinge pin lumen 354 and surface 366 is less than the radial distance between the longitudinal axis of hinge pin lumen 354 and outer surface 364 of boss 362 so that boss forms a shoulder 370. Shoulder 370 interfaces with head 328 to restrict relative translation between tip assembly 332 and camera assembly 306 in the direction of the longitudinal axis of shaft 302.

At the same time, the radial distance between the longitudinal axis of hinge pin lumen 354 and surface 368 is greater than the radial distance between the longitudinal axis of hinge pin lumen 354 and outer surface 364 so that a flexible printed circuit may easily extend from lumen 352, past boss 362 and into camera assembly 306.

Shaft 302 includes lumens (e.g., lumen 310 and lumen 312) that are aligned with and communicate and are aligned with the lumens of tip assembly 332. Shaft 302 may also include additional lumens such as fluid conduit 334 that includes a portion that extends radially through shaft 302 and includes an opening on the side of shaft 302. Preferably, fluid conduit 351 is configured as an injection port and fluid conduit 334 is configured as an evacuation port so that fluid, such as saline, is forced to flow past camera assembly 306 between the injection port and evacuation port. Such a configuration may be used to simplify the use of saline or another fluid to keep the lens of camera assembly 306 clear of debris. Additionally, the fluid conduit used for evacuation also preferably exits the side of shaft 302 so that when optical device is in the first configuration saline or debris may be evacuated before the optical device is removed.

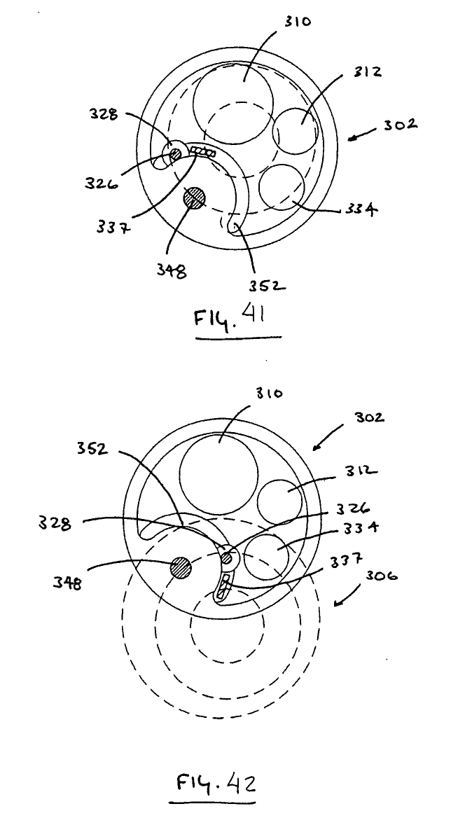

Referring to FIGS. 41 and 42, the relative rotation between camera assembly 306 and shaft 302 will be described. In a first, aligned configuration, shown in FIG. 41, camera assembly 306 (shown in phantom) is aligned concentrically with shaft 302. In that configuration, locating pin 326 is located in a first position along outer surface 364 of boss 362 and flexible printed circuit 337 is disposed adjacent locating pin 326.

Flexible printed circuit 337 may be sealed in each of tip assembly 332 and camera assembly 306 and the portion extending between the sealed location may be sized so that there is slack for free rotation of camera assembly 306. The slack portion of the flexible printed circuit may be located within the counterbored portion of tip assembly 332 when optical device is in the first, insertion configuration. It should be appreciated that the flexible printed circuit may be housed in shrink wrap if desired. Furthermore, stiffeners may or may not be used with the slack portion of the flexible printed circuit so that a desired flexibility may be provided.

In the second, rotated configuration, shown in FIG. 42, camera assembly 306 is rotated relative to shaft 302 about hinge pin 348. In the rotated configuration, locating pin 326 has rotated with camera assembly 306 to a second position along outer surface 364 of boss 362. In the present embodiment, a portion of flexible printed circuit 337 also rotates with camera assembly 306 so that it remains generally adjacent locating pin 326 during rotation. In the present embodiment, the image transmitted from camera assembly 306 may be manipulated so that the image viewed by a user remains as desired. For example, the image may be rotated to counter the physical rotation of camera assembly 306.

Hinge pin 348 is fixedly coupled to camera assembly 306 but is free to rotate within hinge pin lumen 354. In some embodiments, hinge pin 348 may not extend directly to the proximal end of shaft 302 (e.g., through the handle housing), but rather it may have passing through it a thin wire that is anchored in distal tip 324 and on the other end within a control (e.g., knob) in the handle. Rotating the control may turn the wire which in turn may turn distal tip 324. In other embodiments, hinge pin 348 may extend at least to the proximal end of shaft 302 (e.g., through the handle housing) so that it may be easily rotated within hinge pin lumen 354 by a user to rotate camera assembly 306. A dial, knob or other control member may be coupled to hinge pin 348 that is affixed to the handle housing or extends through a side wall of shaft 302, so that hinge pin may be rotated easily relative to shaft 302.

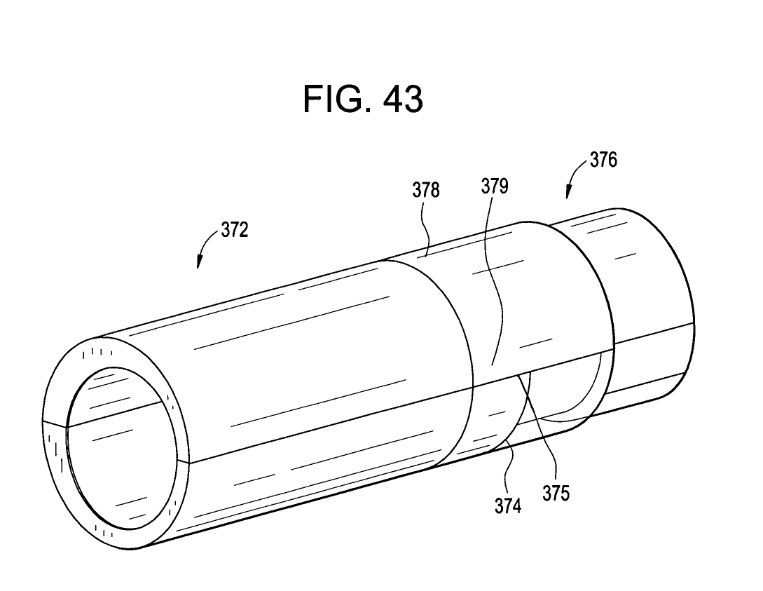

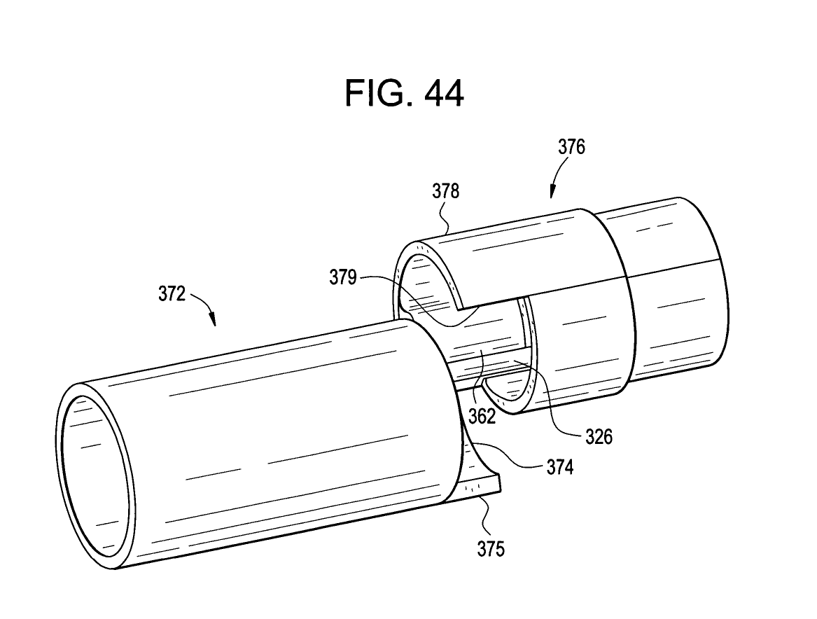

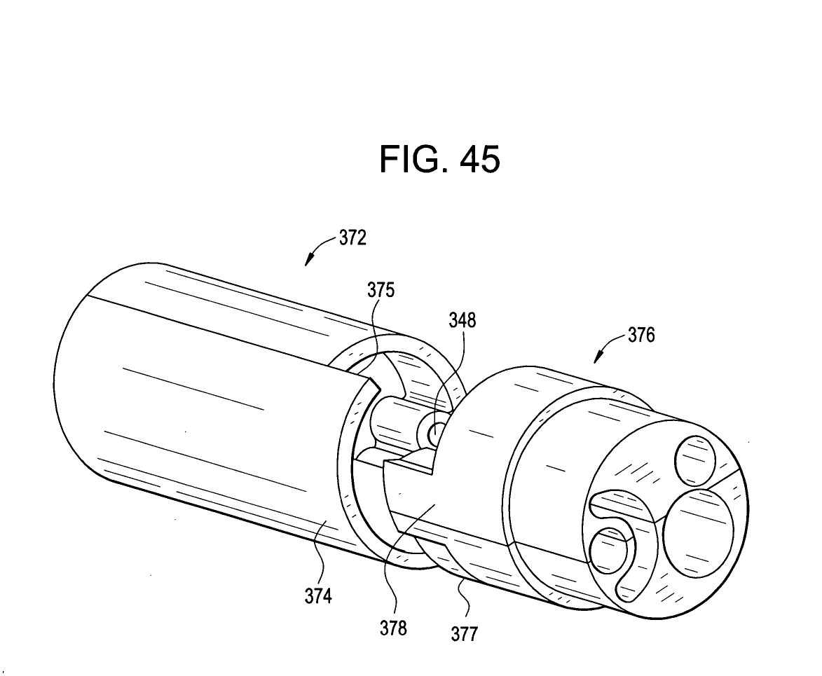

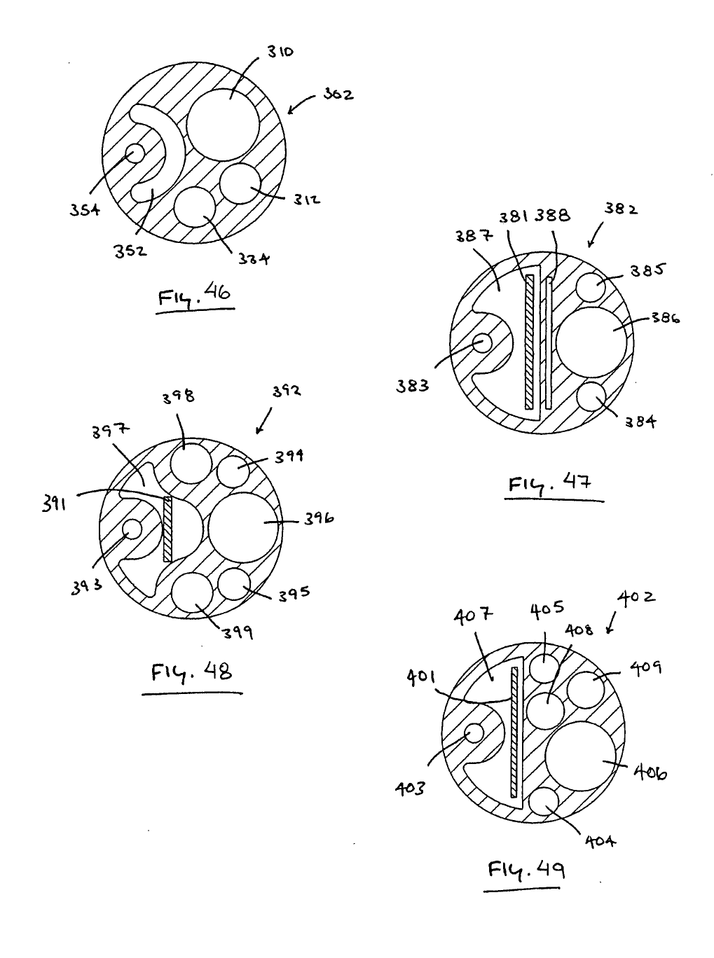

Referring to FIGS. 43-45, optical device 300 is illustrated with an alternative embodiment of the camera housing assembly and the shaft tip assembly. In particular, camera housing assembly 372 includes a circumferential step 374 at its proximal end. Shaft tip assembly 376 includes a complimentary circumferential step 378. As shown in FIG. 43 when optical device 300 is in the first configuration, step 374 and step 378 interlock so that the outer surface of optical device 300 is continuous. However, when optical device 300 is in the second configuration, i.e., when the camera assembly is moved radially outward from the longitudinal axis of the shaft, the steps are rotated away from each other.

The interlocking steps 374 and 378 may be used to provide limit stops for the rotational travel of the camera assembly relative to the shaft. In particular, travel of the camera assembly relative to the shaft in the direction of the first configuration is limited by contact between mating surface 375 of step 374 and mating surface 379 of step 378, as shown in FIG. 43. The travel of the camera assembly relative to the shaft in the direction of the second configuration is limited by contact between a second mating surface 377 of step 374 and the outer surface of shaft tip assembly 376, as shown in FIG. 45.

An advantage of including the complimentary circumferential steps is that the travel of the camera assembly relative to the shaft may be controlled without applying any additional stresses upon the flexible printed circuit. Another advantage is that contact between the mating surfaces in the first configuration of optical device 300 and the contact between the mating surface and the outer surface of the shaft tip provides tactile feedback to the user when the camera assembly is rotated to the ends of travel.

The shaft of the optical device may have any desired configuration that provides any desired combination and orientation of fluid conduits, working channels, stiffener lumens and lumens configured to receive circuitry or wires. Various exemplary embodiments of an elongate body of the shaft are illustrated in FIGS. 46-49. All of the shafts generally include lumens that communicate with and are aligned with lumens of a tip assembly.

As shown in FIG. 46, shaft 302 includes lumens that communicate and are aligned with lumens of tip assembly 332. As described above, shaft 302 includes working channel 310, fluid conduits 334 and 312, lumen 352 for the flexible printed circuit and hinge pin lumen 354.

The shaft may include additional lumens configured for a variety of uses. For example, the shaft may include additional lumens, such as one or more additional fluid conduits and/or stiffener lumens. Referring to FIG. 47, shaft 382 includes hinge pin lumen 383, fluid conduits 384 and 385, working channel 386, lumen 387 for a flexible printed circuit 381, and stiffener lumen 388. In the present embodiment, fluid conduits 384 and 385 are disposed adjacent to and on opposite sides of working channel 386. Additionally, stiffener lumen 388 is rectangular and is disposed between working channel 386 and lumen 387.

Referring now to FIG. 48, shaft 392 includes hinge pin lumen 393, fluid conduits 394 and 395, working channel 396, lumen 397 for a flexible printed circuit 391, and stiffener lumens 398 and 399. Fluid conduits 394 and 395 are disposed adjacent to and on opposite sides of working channel 396 and stiffener lumens 398 and 399 are disposed adjacent respective fluid conduits 394, 395.

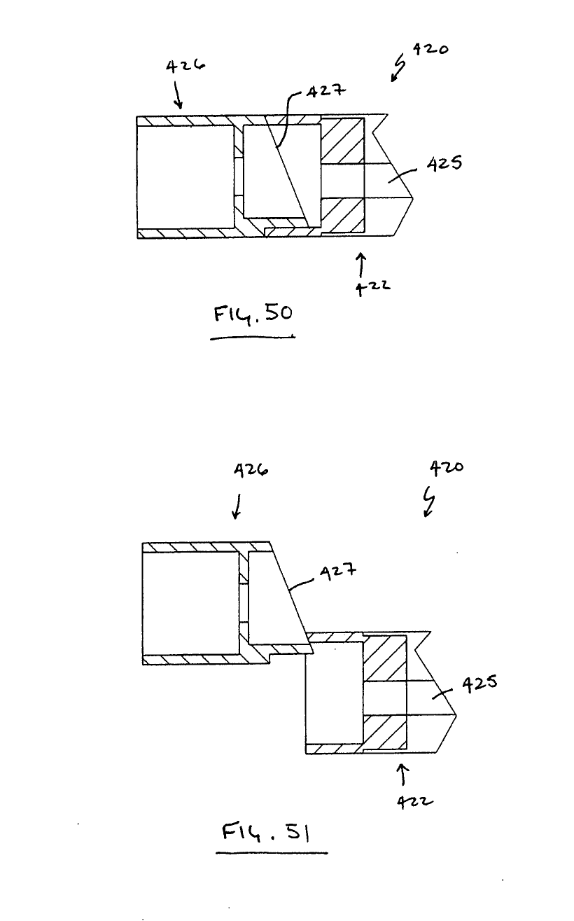

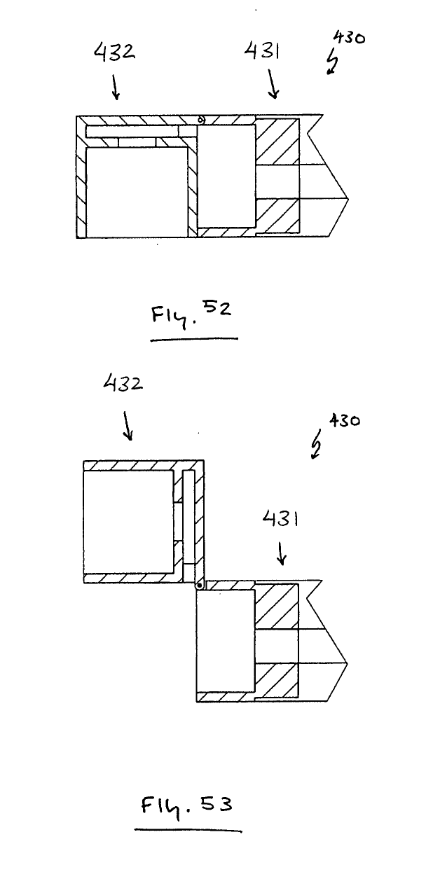

Another embodiment, shaft 402, is illustrated in FIG. 49. Shaft 402 includes hinge pin lumen 403, fluid conduits 404 and 405, working channel 406, lumen 407 for a flexible printed circuit 401, and stiffener lumens 408 and 409. It should be appreciated that any number and type of lumens may be included and the lumens may be any desired shape. It should further be appreciated that the lumen for the flexible printed circuit may be configured so that the flexible printed circuit is not required to rotate relative to the shaft with camera assembly 306. Instead a flexible hinge portion may be included on flexible printed circuit that allows the camera assembly 306 to rotate relative to the shaft. In still other embodiments, a shaft may be provided that includes a working channel capable of simultaneously functioning as a fluid conduit (e.g., saline channel). To facilitate this dual function, the shaft may be provided with a non-circular (e.g., square, rectangular or oval) cross section. At least one corner of the channel may be defined non-circularly. Thus, when an instrument is inserted into the working channel, the corner(s) of the working channel are still unobstructed and thus permit the simultaneous flow of fluid.

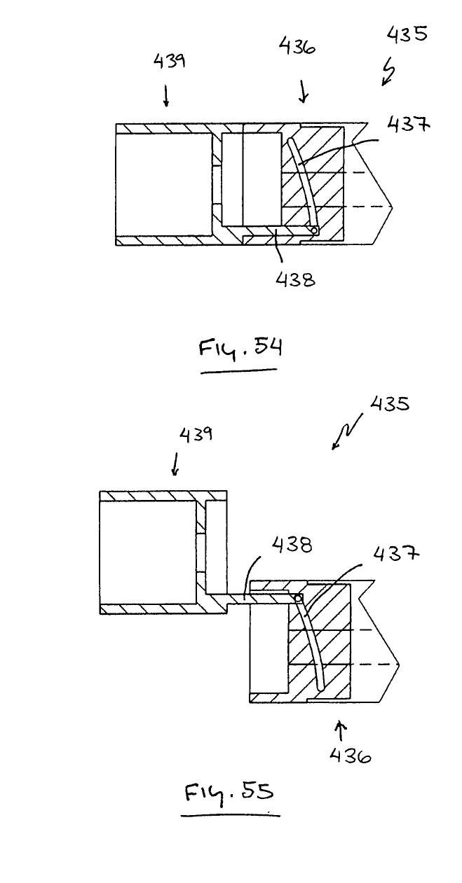

Referring to FIGS. 50 and 51 another embodiment of an optical device that includes a movable camera assembly is illustrated. Optical device 420 includes camera assembly 426 that is movably coupled to a distal end of shaft 422.

Camera assembly 426 includes a ramp surface 427 that is configured to slide along the distal edge of shaft 422 as camera assembly 426 is advanced distally relative to shaft 422. Camera assembly 426 may be advanced using a push/pull wire (not shown) that extends from camera assembly 426 and through shaft 422 that is controlled by a user of optical device. One or more spring members (not shown) may be used to urge camera assembly 426 radially outward from the longitudinal axis of shaft 422 as camera assembly 426 is advanced. Conversely, as camera assembly 426 is retracted, the interface between ramp surface 427 and the edge of shaft 422 causes camera assembly 426 to move radially inward toward the longitudinal axis of shaft 422 against the force of the spring members. Because the camera assembly orientation remains constant, the camera assembly may be used through all stages of use without requiring manipulation of the transmitted image.

The spring members may be any type of spring, for example a coil spring or leaf springs. In an embodiment, the spring members are a pair of leaf springs that form a hoop when they are extended and are flattened when they are compressed. In such an embodiment, the spring members are selected so that the hoop has a diameter that is larger than the diameter of working channel 425 extending through optical device 420 so that the spring members do not impede working channel 425.

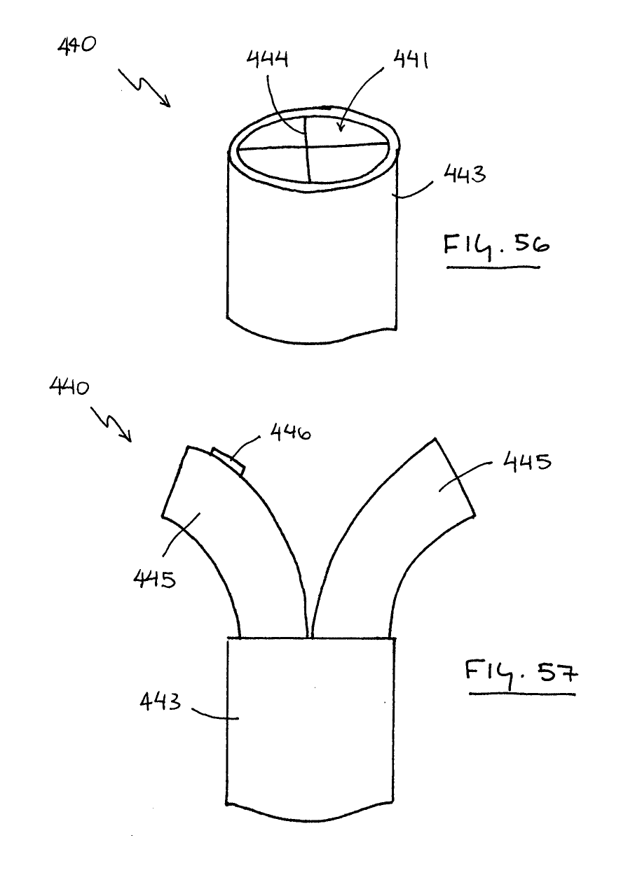

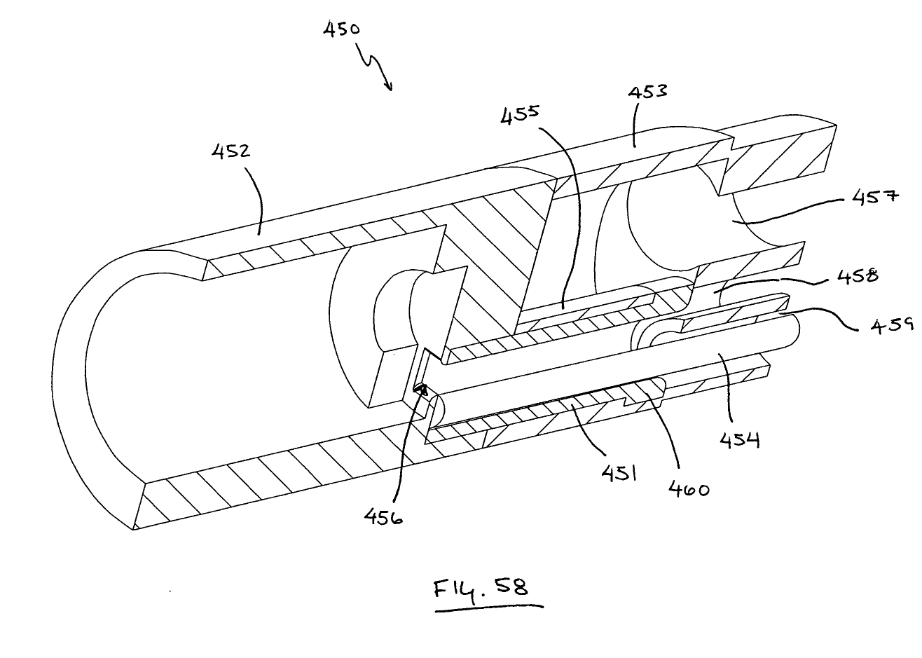

Referring to FIGS. 52 and 53, optical device 430 will be described. Similar to the previously described embodiments, optical device 430 includes camera assembly 432 that is movably mounted to a distal end of shaft 431. In the present embodiment, camera assembly 432 is hinged with shaft 431 so that camera assembly 432 is rotated relative to shaft 431 along an axis that is perpendicular to the longitudinal axis of shaft 431. As a result, during insertion of optical device 430, the optical axis of camera assembly 432 is angled relative to the direction of advancement. Camera assembly 432 may be rotated using a push/pull wire or cable, or any other control mechanism.

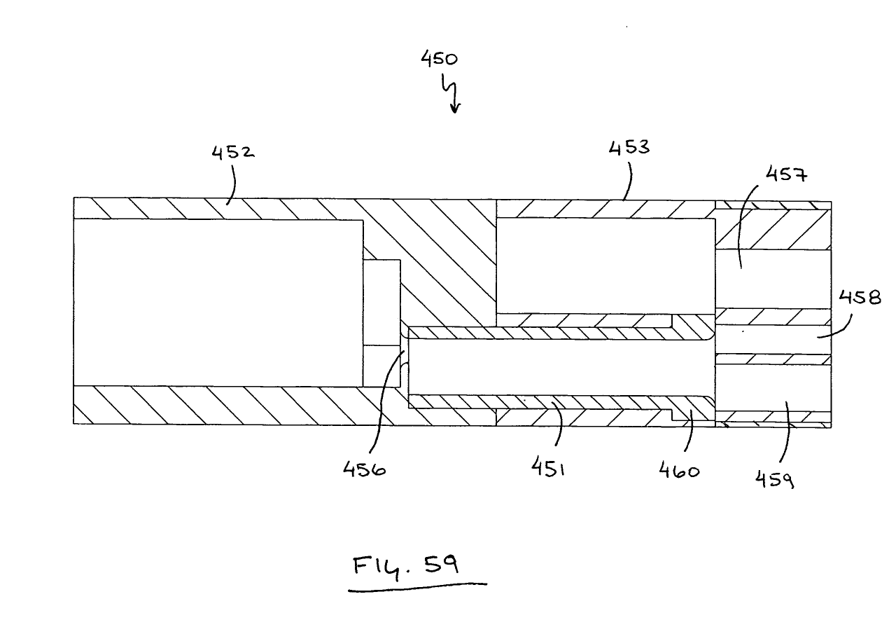

A further alternative embodiment of an optical device having a movable camera assembly is illustrated in FIGS. 54 and 55. Optical device 435 includes camera assembly 439 that is movably coupled to shaft 436 so that it translates radially outward from the longitudinal axis of shaft 436 as camera assembly 439 is advanced distally relative to shaft 436. The motion of camera assembly 439 is limited by one or more rails 437 that may be embedded on the inside or outside of shaft 436. Camera assembly 439 includes at least one arm 438 that engages rail 437 so that arm moves along a predefined path defined by rail 437. A push/pull wire or cable or any other force transmission mechanism may be utilized to move camera assembly 439 relative to shaft 436.

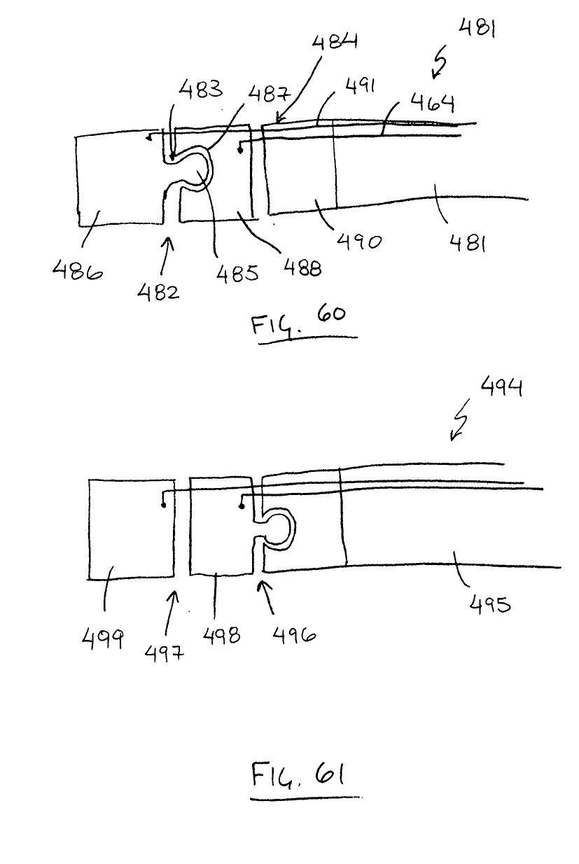

A still further alternative embodiment of an optical device having a movable camera assembly is illustrated in FIGS. 56 and 57. Optical device 440 includes a shaft member 441 and an elongate tube 443 defining a lumen that slidably receives shaft member 441.

Shaft member 441 includes at least one longitudinal slit that extends proximally from the distal end of shaft member 441. Longitudinal slit 444 results in the distal portion of shaft member 441 to be divided into petals 445 that are configured to splay radially outward when they are advanced out of a distal end of tube 443.

In the present embodiment, at least one camera assembly 446 is included in optical device 440 and coupled to an inner surface of a petal 445 so that when petals 445 are in a closed position, camera assembly 446 is at least partially concealed within shaft member 441. It will be appreciated that any number of longitudinal slits 444 and camera assemblies 446 may be provided. For example, in another embodiment, a pair of longitudinal slits 444 are provided thereby defining four petals 445 and a camera assembly 446 is provided on each petal. In such an embodiment, camera assemblies 446 may be disposed on petals 445 so that when petals 445 are in the closed configuration, camera assemblies 446 are generally aligned longitudinally with respect to each other. In an embodiment, a first camera assembly 446 disposed on a first petal 445 is located furthest distally, a second camera assembly 446 disposed on a second petal 445 is located proximal of the first camera assembly. A third camera assembly 446 disposed on a third petal 445 is located further proximal of the second camera assembly and finally a fourth camera assembly 446 disposed on a fourth petal 445 is located further proximal of the third camera assembly.

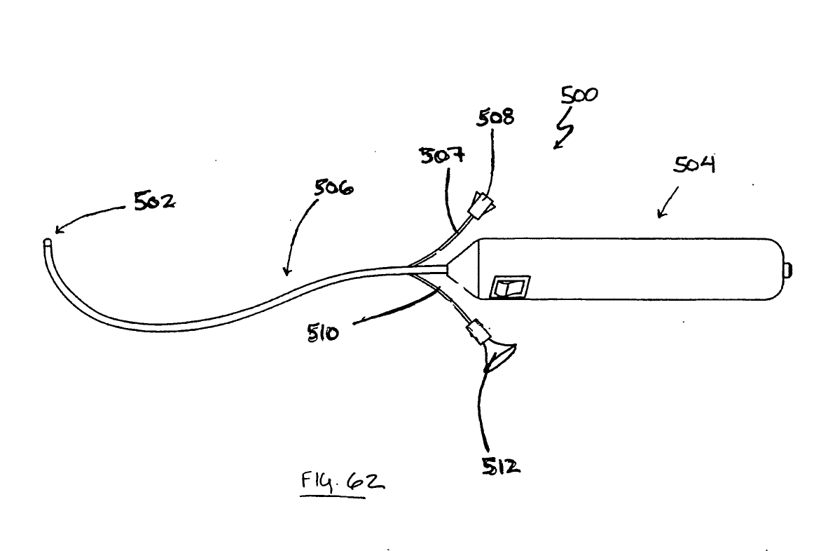

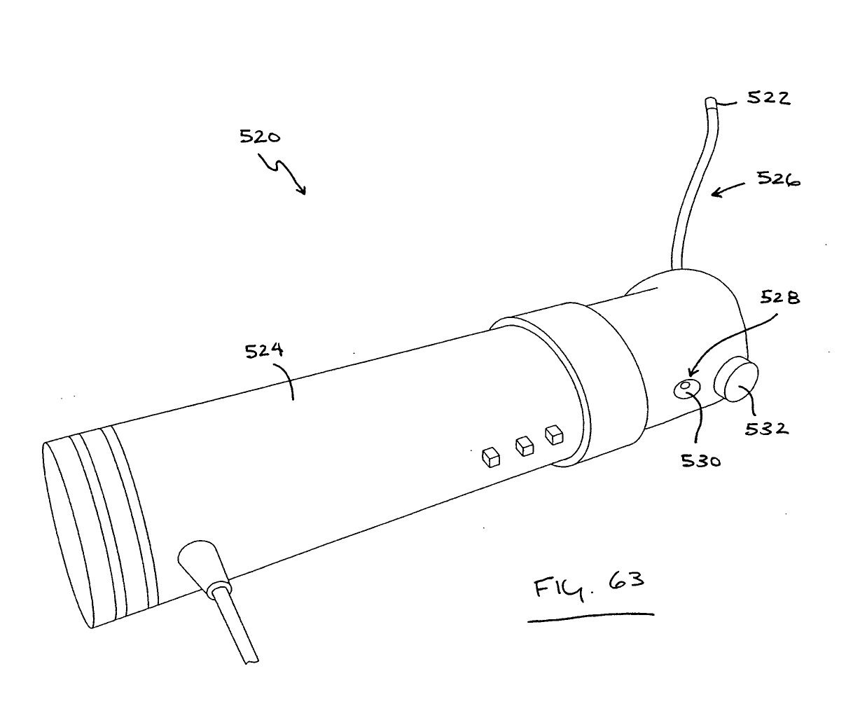

Referring to FIGS. 58 and 59 another embodiment of an optical device having a movable camera assembly will be described. In the present embodiment, a rotating tube 451 provides a rotating interface between camera housing 452 and shaft tip 453. A hinge pin 454, or control wire, extends from a handle end of optical device 450 and through rotating tube 451 and provides control over rotation of camera housing 452 relative to shaft tip 453.

Camera housing 452 has a structure similar to embodiments previously described. In particular, camera housing 452 is a generally tubular member that is sized to at least partially enclose a camera assembly (not shown) and includes a proximal aperture 456 that allows circuitry, such as electrical conductors to pass from shaft tip 453, through camera housing 452 and to the camera assembly.

Shaft tip 453 is fixedly coupled to a distal end of a shaft and may be constructed as a monolithic piece or assembled from multiple components. In the present embodiment, shaft tip 453 includes at least a working channel 457, lumen 458 that is configured to receive a portion of a flexible printed circuit or wires and hinge pin lumen 459, all of which extend through a proximal portion of shaft tip 453 and terminate at a counterbored distal portion of tip 453. Shaft tip 453 also includes boss 455 in the counterbored distal portion.

Rotating tube 451 is an elongate tube that is fixedly coupled at a distal end of tube 451 to camera housing 452. A proximal portion of tube 451 extends through an aperture defined by boss 455 and flange 460, which is included on the proximal end of rotating tube 451, interfaces with a proximal edge of boss 455. The interface between flange 460 and boss 455 retains camera housing 452 in a longitudinally fixed position relative to shaft tip 453 while allowing camera housing 452 to rotate relative to shaft tip 453 about an axis defined by tube 451.

The size and position of rotating tube 451 relative to shaft tip 453 are selected so that the central lumen of tube 451 is in communication with lumen 458 and hinge pin lumen 459. As a result, circuitry extending distally out of lumen 458 and the portion of hinge pin 454 extending distally out of lumen 459 are routed through the central lumen of tube 451. The distal end of hinge pin 454 is fixedly coupled to camera housing 452 and rotatable within the shaft and shaft tip 453 so that the position of camera housing 452 relative to the shaft and shaft tip 453 may be easily manipulated.

Interlocking circumferential steps, as described above, may be included on camera housing 452 and shaft tip 453. The interlocking steps provide rotational limit stops so that the rotational travel of camera housing 452 relative to shaft tip 453 may be easily limited if desired.

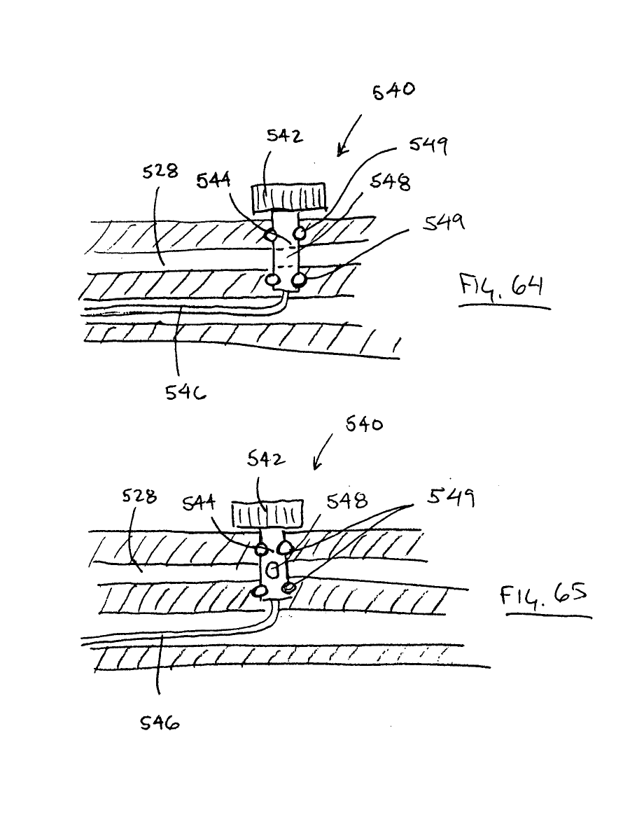

It should be appreciated that the optical device may utilize a hinge joint between the shaft and camera assembly and a radially movable camera assembly in combination. For example, a rotating hinge pin may also be used as a push/pull wire for a hinge joint. Alternatively, both a rotating hinge pin and a push/pull wire may be incorporated. Referring to FIGS. 60 and 61 embodiments of an optical device including both a hinge joint and a rotating joint will be described. First referring to FIG. 60, optical device 480 includes a hinge joint 482 that is located distal of a rotating, or movable, joint 484. As shown, hinge joint 482 is constructed from a hinge member 483 similar to that shown in FIGS. 26-29. In particular hinge member 483 includes a cylindrical projection 485 extending from camera housing 486 that is received in a cylindrical socket 487. However, in the present embodiment, cylindrical socket 487 is included in an intermediate link 488 rather than a shaft tip.

Hinge member 483 is actuated by pull wire 491. Pull wire 491 slidably extends through lumens included in shaft 481 and intermediate link 488 and is fixedly coupled at its distal end to camera housing 486. Similar to the previously described embodiments, pushing and/or pulling of pull wire 491 causes hinge member 483 to bend such that camera housing 486 rotates relative to shaft 481 along an axis that is generally perpendicular to the longitudinal axis of shaft tip 490. It should be appreciated that although hinge member 483 is shown with a cylindrical projection inserted into a cylindrical socket, any type of hinge member may be incorporated.

Movable joint 484 is configured so that camera housing 486 may be rotated relative to shaft 481 about an axis that is parallel and spaced from the longitudinal axis of shaft tip 490. That relative motion allows optical device 480 to be converted between a first configuration in which camera housing 486 and intermediate link 488 are coaxially aligned with shaft tip 490 and a second configuration in which camera housing 486 is moved radially outward from the longitudinal axis of shaft tip 490. Any movable joint that allows that motion may be incorporated such as those previously described.

Movable joint 484 may be actuated by a hinge pin 491. Hinge pin 491 rotatably extends through a lumen in shaft 481 and its distal end is fixedly coupled to intermediate link 488 so that rotation of hinge pin 491 is transmitted to and causes rotation of intermediate link 488 and camera housing 486 relative to shaft 481.

Referring to FIG. 61, optical device 494 includes hinge joint 496 that is disposed proximal of movable joint 497. The structures of hinge joint 496 and movable joint 497 are generally identical to those described above with respect to optical device 480 and will not be further described.

However, it should be appreciated that a single control wire may be used to control both hinge joint 496 and movable joint 497. For example the control wire may be rotatably extended through shaft 495 and intermediate link 498 and fixedly coupled to camera housing 499. As a result, rotation of the single control wire causes camera housing 499 to move relative to intermediate link 498 and pushing and/or pulling the single control wire causes hinge joint 496 to bend.

As previously described, various embodiments include access, or working channels, that are sized so that surgical devices may be advanced through the lumen to a target location. It should be appreciated that the size of the shaft of the optical device is preferably selected so that it is as small as possible while allowing for the desired lumen configuration. As a result, the working channel is generally very small and it is desired to shape the proximal end of the channel to ease insertion of a surgical device.

Referring to FIGS. 62 and 63 embodiments of an optical device including a working channel will be described. With respect to FIG. 62 optical device 500 will be described. In general, optical device 500 includes camera assembly 502, handle 504 and shaft 506 that extends between camera assembly 502 and handle 504. Optical device includes fluid conduit 507 that terminates on the proximal end at fluid connector 508, such as a Luer fitting. Optical device 500 also includes working channel 510 that terminates at a tapered proximal opening 512. It should be appreciated that tapered proximal opening 512 may be provided by a funnel-shaped feature that is integrated into working channel 510 or removably attached to the proximal end of working channel 510.

Referring to FIG. 63, another embodiment, optical device 520, will be described. Optical device 520 generally includes camera assembly 522, handle 524 and shaft 526 that extends between the camera and the handle. In this embodiment, handle 524 is oriented at an angle with respect to shaft 526 and as a result, working channel 528 may extend linearly through a proximal end portion of shaft 526. Working channel 528 includes a tapered proximal opening 530 that is molded into the proximal end portion of shaft 526.

Optical device 520 is configured so that camera assembly 522 may be moved radially outward from the longitudinal axis of shaft 526 and control feature 532 is provided on the proximal end portion of shaft 526 to control that motion.

Control feature 532 may be adapted to provide a closure for working channel 528 that is operated while the position of camera assembly 522 relative to shaft 526 is manipulated. Referring to FIGS. 64 and 65 an embodiment of a control feature 540 including a closure mechanism will be described. Control feature 540 includes handle portion 542 and body portion 544. Handle portion 542 extends from the optical device so that a user may manipulate control feature 540. For example, handle portion 542 is a knob that allows a user to turn control feature 540.

Body portion 544 is generally cylindrical and extends from handle portion 542 and is coupled to control wire 546 that is used as described in previous embodiments to actuate a movable, or rotatable joint. In the present embodiment, control wire is rotated about its longitudinal axis in response to rotation of handle portion 542.

Aperture 548 extends through body portion 544 and is oriented so that control feature 540 may be rotated to align aperture 548 with working channel 528. As shown in FIG. 64, in a first position of control feature 540, aperture 548 is aligned with working channel 528. However, when control feature 540 is rotated by 90 degrees, as shown in FIG. 65, body portion 544 blocks working channel 528. One or more sealing members 549, such as o-rings, bushings or other seals, may be provided so that fluid passing through working channel 528 is prevented from entering the space between body portion 544 and shaft 526. Although body portion 544 is shown generally perpendicular to working channel 528, it should be appreciated that body portion 544 may be oriented at any angle with respect to body portion 544 if desired.

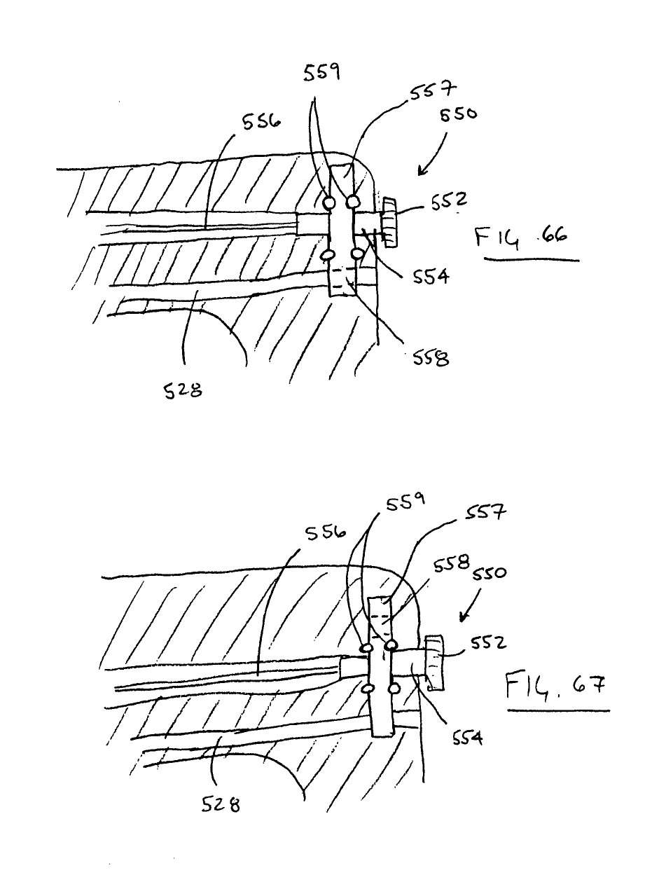

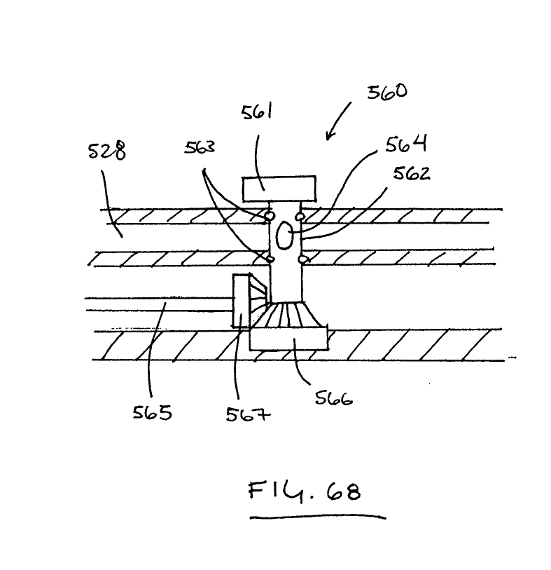

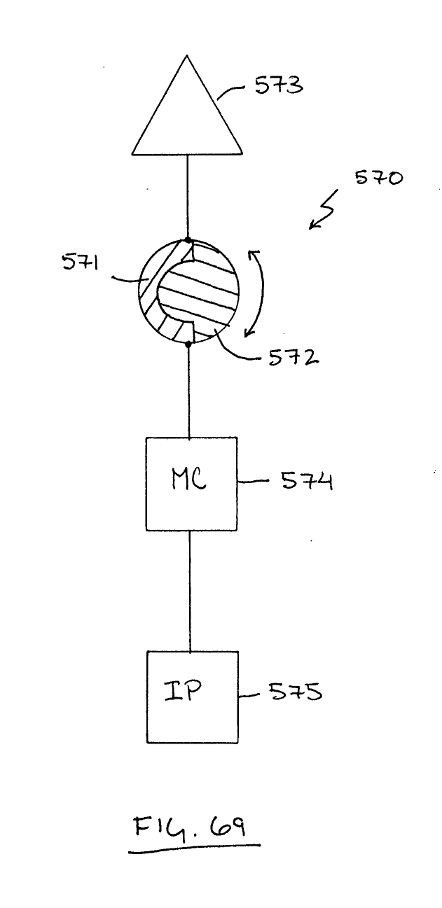

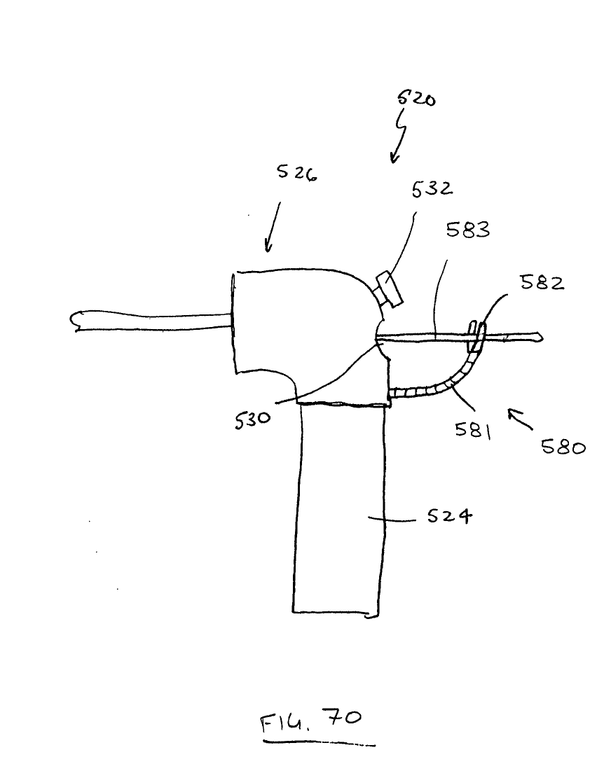

Another embodiment of a control feature including a working channel closure mechanism is illustrated in FIGS. 66 and 67. Control feature 550 also includes handle portion 552, body portion 554 and sealing members 559 and is configured to rotate a control wire 556. However, in the present embodiment, a closure arm 557, or disk, extends perpendicularly from body portion 554 and selectively blocks working channel 528. As shown, closure arm 557 includes a portion with an aperture 558, however, it should be appreciated that a section of closure arm 560 may be removed, rather than providing an aperture, so that working channel is open when closure arm 557 is rotated away from working channel 528.

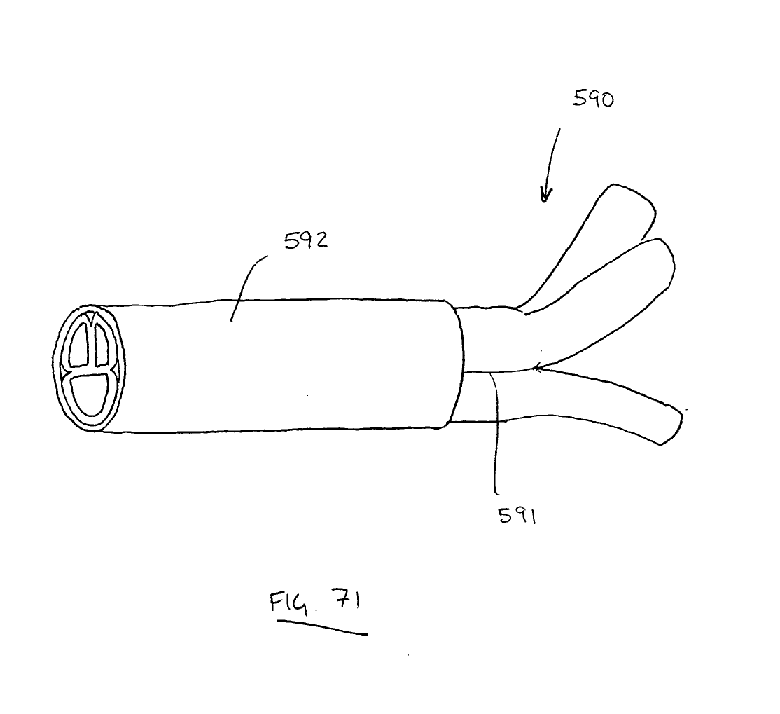

Referring to FIG. 68 another embodiment of a control feature including a working channel closure mechanism will be described. Control feature 560 includes handle portion 561, body portion 562, sealing members 563 and aperture 564. Control feature 560 is rotatably coupled to a shaft of an optical device so that aperture 564 is generally aligned with a working channel 528.

In the present embodiment, a control wire 565 is coupled to control feature 560 through a geared interface. In particular, an input gear 566 is fixedly coupled to an end of control feature 560 opposite handle portion 561. Input gear 566 meshes with an output gear 567 that is fixedly coupled to a proximal end of control wire 565. Rotation of control feature 560 is converted into rotation of control wire 565 by the geared interface. The size and tooth count of each gear are selected to provide any desired mechanical advantage. For example, in an embodiment, input gear 566 and output gear 567 are selected so that a 90 degree rotation of control feature 560 causes control wire 565 to rotate approximately 180 degrees.

Additionally, the control features may provide visual indication of the position of the camera assembly and/or the aperture of the control feature. For example, indicia may be provided on or adjacent the handle portion that indicate the amount of rotation of the camera assembly relative to the shaft. Furthermore, body portion may include a colored portion that is easily visible when access to the working channel is blocked. For example, some or all of the body portion, or closure arm, of the control feature may be colored red so that when access to the working channel is prevented, the user will see the colored portion. It will be appreciated that although all of the control features described above are combined with a working channel closure mechanism the closure mechanism need not be included. For example, the closure mechanisms described above may be employed in embodiments of the optical device that do not include a working channel.

As a further alternative, a switch may be built into the control feature so that the switch provides continuity or discontinuity at a desired rotational position of the control feature. The switch may be electrically coupled to a visual or audible indicator. For example, the switch may be coupled to an LED that is lit when access through the working channel is either permitted or prevented.

In a still further embodiment, such a switch may be used to rotate the image output of the optical device as schematically illustrated in FIG. 69. In particular, switch 570 includes a conductive portion 571 and a non-conductive portion 572. Switch 570 selectively electrically couples power source 573 to a microcontroller 574 and an image processor 575. The microcontroller is configured so that it detects continuity across switch 570 and passes instructions to image processor 575 to display the image in a desired orientation. In particular, it is desirable that the image output remain in a constant orientation regardless of the rotated position of the camera assembly relative to shaft. The continuity of switch 570 corresponds to a rotational position of camera assembly relative to the shaft so that the microcontroller can provide commands to the image processor to maintain the constant orientation.

In the present embodiment, switch 570 provides binary continuity, i.e., it is either fully continuous or fully discontinuous. However, it should be appreciated that any type of switch may be utilized including those that provide progressive feedback. For example, the control feature may be coupled to an adjustable resistor or a potentiometer so that the image is rotated in increments corresponding to increments of rotation of the camera assembly relative to the shaft.

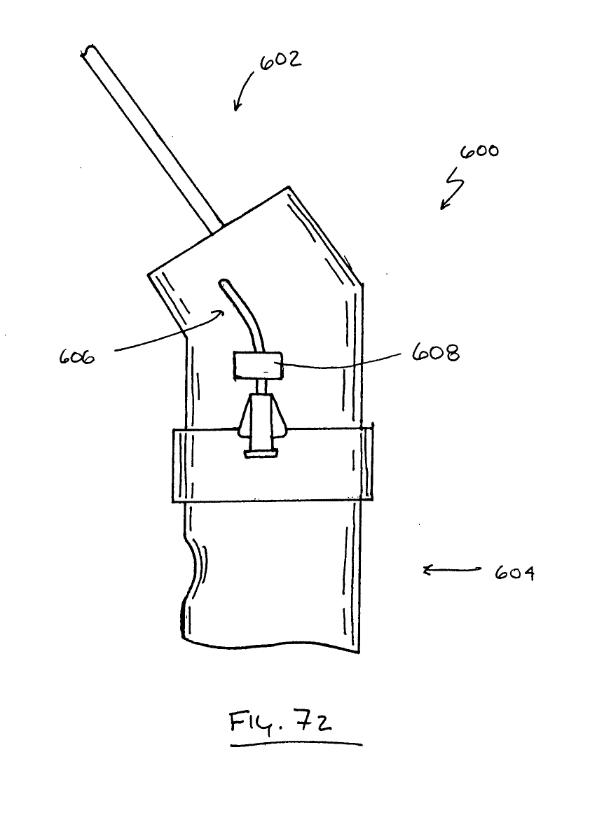

A surgical device holder 580 may also be coupled to the proximal end portion of shaft 526 or handle 524 of the optical device, as shown in FIG. 70. Such a holder may be provided so that a user is not required to hold both the optical device and the surgical device at the same time. Holder 580 includes a shapeable body portion 581 and a clip portion 582. The body portion 581 may be a shapeable gooseneck or any other shapeable elongate structure. Clip portion 582 may be a universal clip that is configured to releasably hold a wide variety of surgical devices or it may be specifically tailored to a surgical device.

As described previously, the shaft of the optical device may have many different configurations based on the desire to provide fluid conduits, working channels, stiffening channels, etc. Depending on the configuration, it may be necessary to provide portions of those lumens that separate from the main body of the shaft. Adapters may be used that provide extensions of the lumens separate from the main body of the shaft, such as those shown in FIGS. 1, 19 and 62 for fluid conduits and/or working channels. However, it may be desirable to provide a shaft construction that obviates the need for separate adapters while still providing access to the lumens.

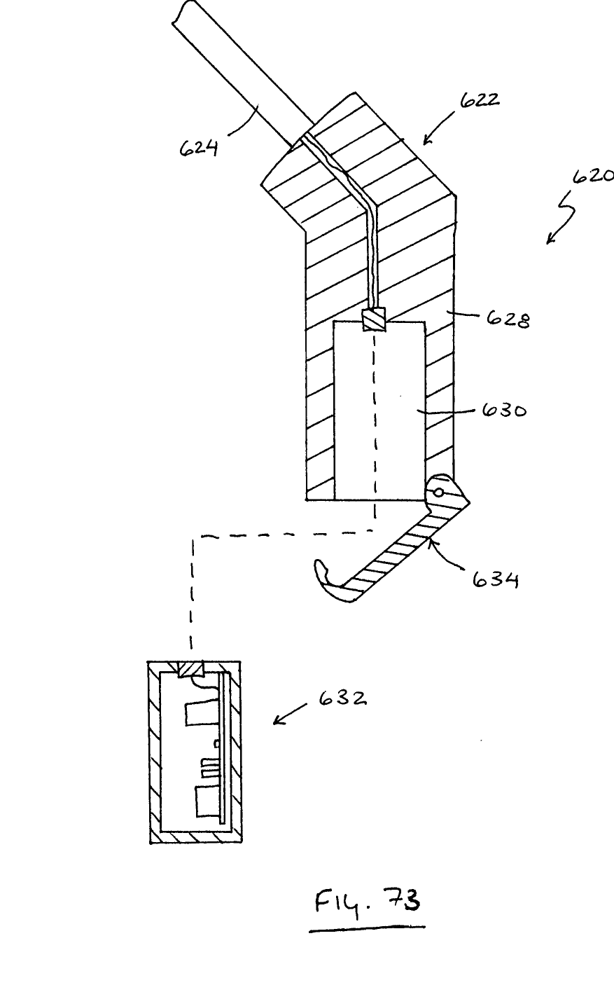

Referring to FIG. 71, a shaft 590 that has a construction that obviates the need for separate adapters will be described. Shaft 590 is generally a multi-lumen elongate extrusion. Lines of weakening 591, such as perforated tear lines or thinned portions of the extrusion, are provided so that portions of shaft 590 may be separated. Lines of weakening 591 are oriented so that each portion of shaft 590 containing a lumen may be separated from the remainder of shaft.

It should be appreciated that lines of weakening 591 may extend the entire length of shaft 590 or for only a portion of shaft 590. In embodiments utilizing lines of weakening extending the entire length, a tear stop feature, such as shrink wrap 592 or any other coating or stop may be included so that separation of the portions of shaft 590 is prevented beyond a desired predetermined location.

In embodiments that include portions of the lumens that separate from the main body of the optical device it may be desirable to include features on the main body that allow those separate portions to be temporarily coupled to the side of the main body. Referring to FIG. 72, optical device 600 includes handle 604 and shaft 602. Shaft 602 includes at least one fluid conduit 606 a portion of which extends away from a proximal portion of shaft 602. A retaining member 608 is provided that is configured to hold the proximal portion of fluid conduit 606 in place when optical device 600 is manipulated. Retaining member 608 may be any feature capable of releasably coupling fluid conduit 606 to a side wall of handle 604 or shaft 602, such as a hook-and-loop fastener, a clip, or a groove that has a width that is less than the outer diameter of fluid conduit 606.

As described above it is often desirable to provide an optical device that includes both disposable and reusable portions. Various embodiments utilizing a shaft portion that is removable from a handle portion have been previously described. Another configuration that may alternatively be incorporated into any of the previously described embodiments includes an outer body that includes both shaft and handle portions and a removable cartridge that, during use, is completely enclosed by the handle portion.

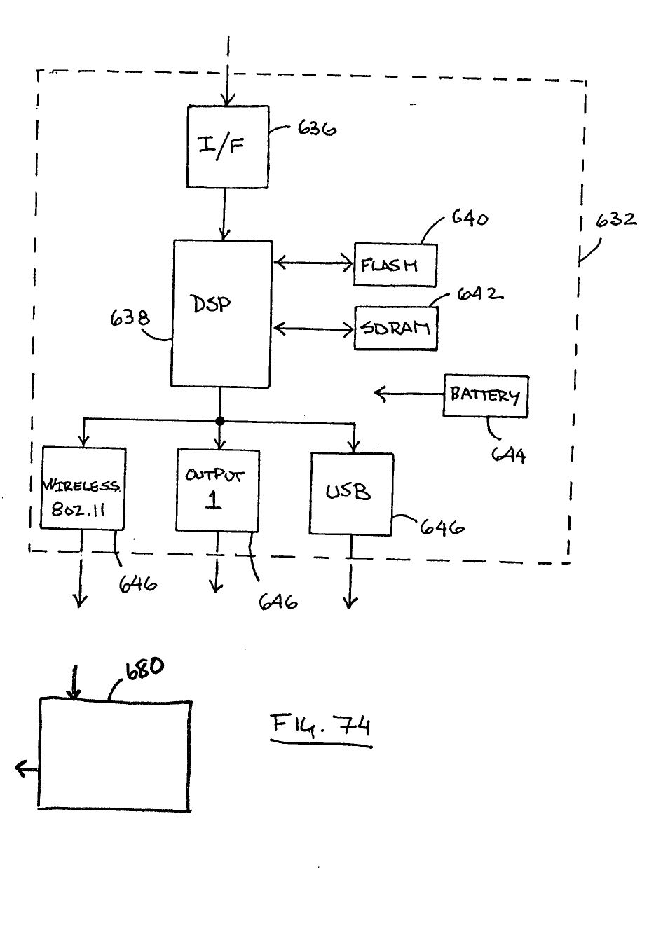

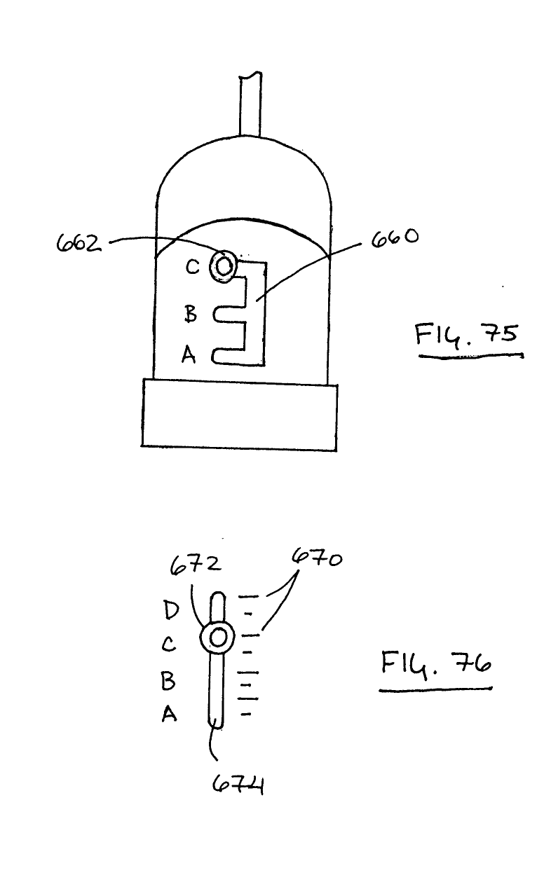

Referring to FIGS. 73 and 74 an embodiment including the removable cartridge configuration will be described. Optical device 620 includes an outer body 622 that includes shaft portion 624 that extends between a camera assembly and handle portion 628. The camera assembly and shaft portion 624 are constructed as any of the previously described embodiments. Handle portion 628 is integral with shaft portion 624 and extends proximally from shaft portion 624. A cavity 630 is provided in handle portion 628 that is sized to receive removable cartridge 632.

A cavity access member 634 is provided that allows handle portion 628 to be opened and closed so that cartridge 632 may be inserted into or removed from cavity 630. In the present embodiment, access member 634 is a hinged lid disposed on a proximal end of handle portion 628. The hinged lid includes a pivoting connection to handle portion 628 and a latch mechanism. Access member 634 is also configured to fluidly seal cavity 630 when it is in a closed configuration so that cartridge 632 is not exposed to fluids during use of optical device 620. Any sealing mechanism, such as a flexible gasket, may be included in access member 634 and/or handle portion 628 to provide the fluid seal.

Cartridge 632 is sized to fit within cavity 630 and includes a battery, processing microchips, support microchips and at least one electrical connector. Generally, cartridge 632 contains an input connector, a sensor interface 636 for receiving information from the camera, an image processing engine 638, a storage module 640, a temporary storage module 642, a power supply 644 and at least one output module 646. In an embodiment, as shown in FIG. 74, cartridge 630 houses a sensor interface, a digital signal processor, flash memory, SDRAM, a battery and at least one output module such as a wireless module, an NTSC/PAL module and/or a USB module. Including the components in a reusable cartridge allows the expensive electronic components to be reused while avoiding their exposure to body fluids and any other contaminants.

In some embodiments, device 680 may be provided that receives an analog video signal (e.g., NTSC signal) broadcast by a transmitter included in the optical device (e.g., broadcast by transmitter 646 (FIG. 74)). Device 680 may be a wireless receiver that converts the analog signal for output via a USB port to, for example, a computer (e.g., personal computer). Wireless receiver 680 may demodulate the NTSC signal, digitize the NTSC signal, and convert the digital data into a protocol suitable for receipt by the computer. The conversion may be performed such that the computer may not need a dedicated or additional driver to process the signal received via the USB port. For example, the signal may be compliant with the wave division multiplexing (“WDM”) model for video transmission.

As described above, the optical device generally includes a camera assembly that is movable relative to a shaft. In some of the described embodiments, the camera assembly is rotated relative to the shaft about an axis that is perpendicular to the shaft. In other described embodiments the camera assembly is rotated relative to the shaft about an axis that is parallel to a longitudinal axis of the shaft so that the camera assembly may be moved radially outward to open a distal end of conduits extending through the shaft, such as a working channel or fluid conduit. Control features are provided that allow a user to manipulate the position of the camera assembly relative to the shaft. Temporary locking mechanisms may be included that allow the position of a control feature to be temporary locked in place relative to the shaft so that the position of the camera relative to the shaft is held constant. In embodiments utilizing rotating control features, friction with sealing members, ratchet assemblies and/or set screws, such as thumb screws, may be included to selectively restrict rotation of the control feature. In embodiments utilizing sliding control features, a slot 660 within which control feature 662 is configured to slide, may be shaped to provide locking locations. For example, as shown in FIG. 75, control feature 662 may be slid between positions A, B and C, each of which corresponds to a position of a camera assembly relative to the shaft.

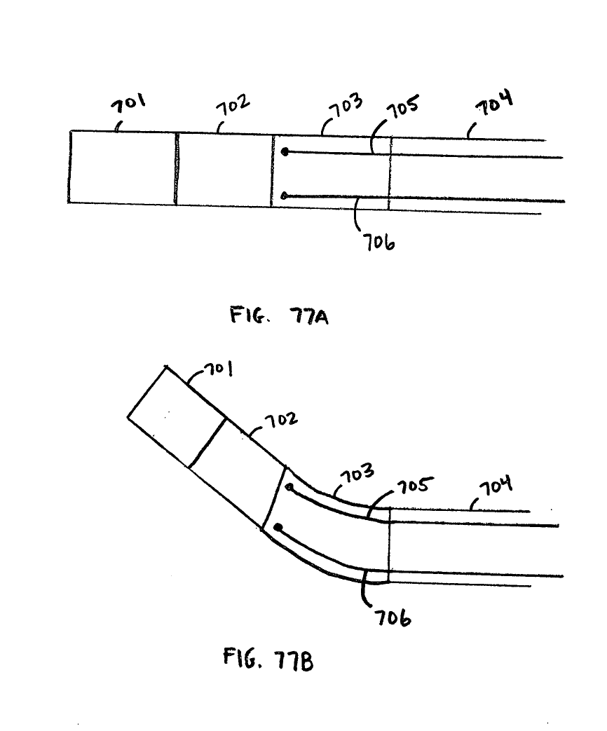

Indicia may also or alternatively be provided so that the position of the camera assembly may be easily determined. For example, markings may be included on and adjacent a control feature so that a visible indication of the position of the camera is provided. Referring to FIG. 76, indicia 670 are provided adjacent control feature 672. As control feature 672 is slid within slot 674, the individual indicia 670 nearest control feature 672 provides a visual indication of the position of the camera assembly.

FIG. 77A shows another embodiment of a shaft for coupling to a distally-positioned camera according to some embodiments of the present invention. Camera housing 701 and proximal tip 702 may be the same as or similar to the camera housing and proximal tip shown in FIGS. 34-36. For example, a locating pin may be provided that extends through proximal tip 702 and into semi-rigid portion 703 of the elongate shaft. Semi-rigid portion 703 may be a generally tubular member made by, for example, plastic extrusion. In some embodiments, semi-rigid body 703 may be about 8-10 mm long, and may have the same diameter (e.g., 5.5 mm) as rigid body 704 of the elongate shaft. In some embodiments, rigid body 704 may extend all the way from semi-rigid body 703 to a handle portion of the optical device. In other embodiments, rigid body 704 may extend only a portion of the distance to the handle and may be coupled to one or more proximal semi-rigid portions. For example, rigid body 704 may be made from stainless steel or other rigid material and may couple to semi-rigid body 703 using an internal connector. In other embodiments, rigid body 704 may be made from the same material as semi-rigid body 703 but may include rigid stiffening bod(ies) disposed therein. Pull wires 705 and 706 may be anchored at, for example, the distal end of semi-rigid body 703. When one of wires 705 and 706 is pulled (e.g., wire 705), camera housing 701 and proximal tip 702 may deflect in the direction of that wire, as shown in FIG. 77B. The bearing of semi-rigid body 703 against rigid body 704 may provide for more reliable deflection of the camera and proximal tip upon actuation of the pull wire.

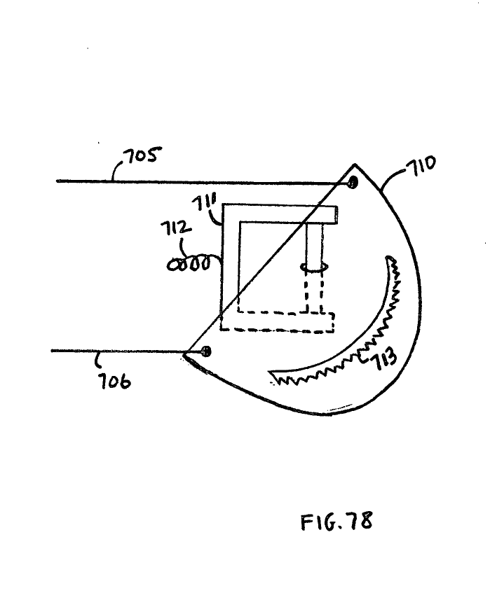

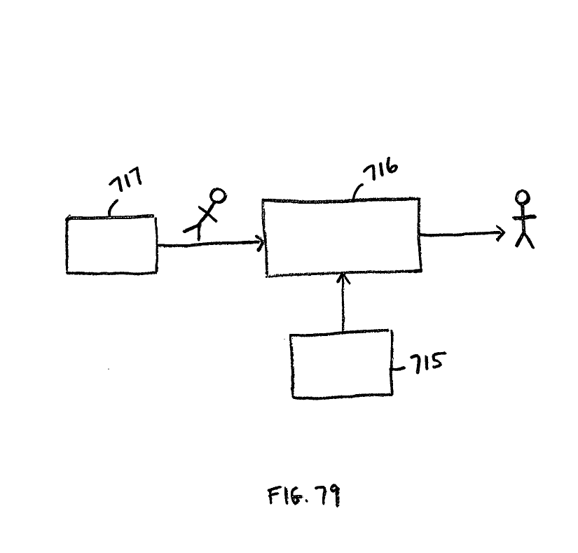

FIG. 78 shows an embodiment of a semi-circular knob 710 for connecting to, for example, pull wires 705 and 706 of FIGS. 77A and 77B. As one wire 705 is pulled a certain amount by a user turning knob 710, knob 710 pushes/releases the other wire 706 by the same amount. Knob 710 may be attached to the handle of the optical device and may be spring loaded through attachment to clevis 711 and spring 712. To enable turning of knob 710, the user may be required to push in knob 710 to cause teeth 713 to disengage from a mating feature in the handle housing. When knob 710 is released, teeth 713 may once again engage the mating feature, thus locking the desired deflection of the control wires in place.

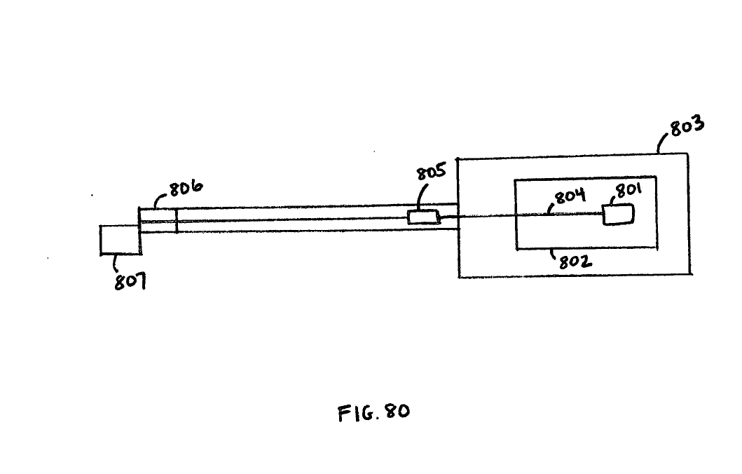

In some embodiments, an optical device in accordance with the present invention may process images received from the distally-positioned camera in order to correct for rotation imparted to the images by rotation of the shaft, camera housing, and or proximal tip. FIG. 79 shows an accelerometer 715, digital signal processor 716, and image sensor 717 according to some embodiments of the present invention. Accelerometer 715 and digital signal processor 716 may be included within the handle housing (e.g., within housing 22 of optical sensor 10 (FIG. 1)), whereas image sensor 717 may be coupled to the distal end of the elongate shaft. In response to accelerometer 715 sensing rotation of the shaft (e.g., 40 degrees clockwise), accelerometer 715 may cause digital signal processor to correct a raw image received from the image sensor for this rotation (e.g., by rotating the image 40 degrees counter-clockwise) and output a corrected image. Thus, when images from the optical sensor are displayed on a display device, the image may always appear right-side up. For example, accelerometer 715 may be a 3-axis accelerometer that determines and isolates the direction of a gravitational force. Once the direction of the gravitational force is known, it is known which way is “down.” That information, coupled with prior knowledge of the relative position of the image sensor to accelerometer 715, indicates which way the sensor is oriented and thus the image can be rotated appropriately to correct for rotation of the device.

FIG. 80 is a simplified view of an optical device that includes a high-powered light-emitting diode (“LED”) within a reusable part of the device, according to some embodiments of the present invention. As shown, LED 801 may be included within reusable cartridge 802 (e.g., cartridge 632 (FIG. 73)), which may be removable from housing 803. In some embodiments, LED 801 may transmit light to the distal end of the shaft by way of incoherent or single core fiber 804. Fiber 804 may coupled to LED 801 by cylindrical mate 805, which may be located within the shaft or handle housing 803. In some embodiments, fiber 804 may extend all the way through the shaft to the distal end of the camera housing. This, however, may require an increase in the diameter of the shaft. In other embodiments, fiber 804 may terminate at the distal face of proximal tip 806, as shown in FIG. 80. In some embodiments, LED 801 may be turned on only when camera housing 807 is rotated axially relative to proximal tip 806. Sensing of such axial rotation and turning on of the LED may be triggered by accelerometer 715 (FIG. 79), also included within cartridge 802. In some embodiments, camera housing 807 may include additional light sources (e.g., light sources 52 (FIG. 3)).

In some embodiments, the optical device may be provided with an input and suitable electronic circuitry for communication with a commercially-available sterilization device. The sterilization device may plug into an input port included within the handle housing of the optical device, and can then be inserted down the working channel. For example, the optical device and the sterilization device may be provided in the same kit (e.g., Tyvek bag). Since some sterilization devices can be used for both fallopian tubes, a second device will not be required to be inserted within the working channel. In some embodiments, the sterilization device may be built entirely or partially into the shaft and/or handle of the visualization device. According to such a design, the working channel of the shaft can be left open for other instruments.

Methods for ob-gyn examinations using the above described devices are disclosed next. One of ordinary skill in the art will appreciate that the methods disclosed herein are equally applicable to the examination of different body cavities, within the appropriate clinical settings and the appropriate preparation of the patient and of the cavity.

During patient intake, a clinical assistant, typically a nurse, records personal data of the patient and medical history, including gravidity, parity, last menstrual period, contraception use, prior abnormal pap smear results, allergies, significant past medical history, medications, prior cervical procedures, and smoking history. The patient is then invited to lay on an examining table in the dorsal lithotomy position, with her legs in stirrups and her buttocks close to the lower edge of the table.

After the vulva is examined for any suspicious lesions, a speculum is placed in the vagina to spread the vaginal walls and provide improved visual access to the clinician. An acetic acid solution is then applied to the cervix to help the clinician assess whether a change in the color or in the vascular pattern of the cervix and vaginal tract are indicative of abnormalities. An iodine solution may also be applied to the cervical area, to help in highlighting areas of abnormality. These preparatory procedures are known in the art and are not detailed herein for the sake of brevity.

The optical device disclosed hereinabove is utilized for the successive steps of the examination method. The shaft of the optical device is inserted into the dilated vaginal canal and an examination of the vaginal canal and of the outer cervical area is performed. The thin and extended shape of the shaft enables the clinician to examine closely all areas of the vaginal canal and of the outer cervical region, including the posterior wall and fornix of the vagina, the anterior and posterior lips of the cervix, and the opening of the uterus. Visual clarity is provided by the light source(s) in the distal region of the optical device, and desired color tones, that highlight with greater clarity certain variations in vaginal and cervical tissue and in vascular pattern, may be adjusted by modulating light sources of different colors to achieve the desired color combination. Close-ups of regions of interest can be provided by actuating a built-in zoom, and the resulting image can be viewed on a video monitor or on a PC, or, in a field situation, on a laptop screen. Because of the dilation of the vagina, the examining clinician typically need not cause the camera tip of the instrument to flex or to rotate in relation to the shaft, but can simply examine lateral areas of the vagina by moving the handle of the instrument as required or by preshaping to shaft to a desired contour.

Therefore, in this phase of the examination, the optical device provides an output comparable to colposcopy, but with a smaller, more agile, and less expensive instrument. The apparatus of the present invention also enables a tailoring of the light colors and a close-up examination of areas that may be difficult to visualize with a colposcope. Because the shaft of the optical device is bendable in a goose-neck fashion, the clinician may provide the shaft with a specific shape to examine more closely and more easily a specific area of interest. The optical device of the present invention is also portable and enables a close-up examination in a field setting, for example, during a home visit, in remote areas where colposcopes are not readily available, or in a military environment. A biopsy sample, when required, may be removed with a separate instrument or with an instrument inserted in a working cannel of the optical device.

When an uterine examination is also required, the shaft of the optical device is inserted in the cervical canal. The diameter of the head of the instrument varies according to the different embodiments, but is typically less than 5 mm, making the instrument suitable for insertion in the cervical canal without the use of a dilation device or even of an anesthetic. A topical anesthetic, such as lidocaine or a cervical block, and/or a dilating device may be employed to reduce discomfort to the patient or when specific anatomic or physiological situations so require. The shapability of the shaft of the optical instrument provides for the shaft to retain pushability, that is, for the shaft to assume a desired contour while retaining a sufficient compressive strength to overcome resistance to insertion into the cervical canal.

Once the optical device has reached the uterine region, the uterus is distended by injecting a fluid known in the art, for example, a saline solution or CO₂. The modes of fluid distribution and removal vary according to the different embodiments of the optical device. For example, to minimize shaft diameter, the distending fluid may be injected through a first opening situated below the camera head and laterally on the shaft, and may be removed by aspiration through a second opening, also situated below the camera head, as shown in FIG. 13. This configuration of the first and second openings provides for a smaller head diameter compared to other devices, in which the openings are situated at the distal tip of the device, adjacent to the camera. This configuration also provides for lesser fluid turbulence at the instrument tip due to ejection and removal of the fluid, providing for a clearer filed of vision than with instruments having fluid ejection (and also fluid removal, when present) adjacent to the camera head. One skilled in the art will appreciate that any of the previously disclosed embodiments may also be utilized in this step in lieu of the embodiment of FIG. 13.

The distending fluid also may be prevented from entering the working channel by the seal disposed at the distal opening of the working channel (for example, the petal-shaped seal described above) in embodiments where the distal opening of the working channel is exposed to the outer environment. In embodiments where the distal opening of the working channel becomes in contact with the outer environment only upon a rotation or a displacement of the camera head, the distending fluid is prevented from entering the working channel because the distal opening of the working channel is not in contact with the fluid until the head of the instrument is rotated or displaced.

Specific areas of the uterus can be visualized by appropriately tilting the camera head and by zooming into the areas of interest. Using the embodiment of FIG. 60 as a non-limiting example, camera housing 486 may be rotated angularly by rotating cylindrical projection 485 to examine different areas of the uterus within the angle of rotation. Camera housing 486 and intermediate link 488 may also be tilted in a radial direction (that is, sideways) to acquire a longitudinal axis parallel to but not coincident with the longitudinal axis of optical device 481, and/or may be rotated in a radial direction and be also tilted angularly along the axis of hinge member 483. One skilled in the art will appreciate that different embodiments will have different rotation movements, according to the construction of each specific embodiment.

If a working channel is provided, the clinician may extract a biopsy sample, or perform other exploratory or surgical procedures, by inserting an instrument in the working channel and by operating the instrument under the visual guidance provided by the camera head. In the embodiment of FIG. 60, used again as an illustrative and non-limiting example, the working channel extends to the distal end of shaft tip 490. Once intermediate link 488 and camera housing 486 have been moved laterally, the surgical instrument may exit the working channel and reach the area of interest. Operation of the surgical instrument may be performed according to techniques known in the art, which are not repeated here for the sake of brevity.

Introduction of the surgical instrument into the working channel may be assisted by providing a tapered proximal opening to the working channel, such as those indicated by reference numerals 512 in FIGS. 62 and 530 in FIG. 63. An endocervical curettage may also be performed, if required in the judgment of the clinician. Methods for extracting biopsy samples and for surgical procedures, as well as for related bleeding control and follow-up procedures, are known in the art, and will not be repeated here for the sake of brevity.

In view of the different features of the embodiments of the apparatus disclosed herein, a person skilled in the art will readily appreciate that different embodiments of the method of use of the optical apparatus of the present invention are also possible, in accordance with the specific features of the optical device in the related embodiments. Such alternative embodiments of the method of use are all within the scope and spirit of the present invention.

Thus it is seen that apparatus for examining a body cavity and methods of use are provided. Although particular embodiments have been disclosed herein in detail, this has been done by way of example for purposes of illustration only, and is not intended to be limiting with respect to the scope of the appended claims, which follow. In particular, it is contemplated that various substitutions, alterations, and modifications may be made without departing from the spirit and scope of the invention as defined by the claims. Other aspects, advantages, and modifications are considered to be within the scope of the following claims. The claims presented are representative of the inventions disclosed herein. Other, unclaimed inventions are also contemplated. The applicant reserves the right to pursue such inventions in later claims.

What is claimed is:

1. An optical apparatus comprising:
a housing including a shaft portion, and a handle portion extending from a proximal end of the shaft portion and defining a cavity that contains an output connector;
a camera assembly coupled to a distal end of the shaft, the camera assembly comprising a camera; and
a removable cartridge receivable within the cavity and including an input connector matable with the output connector, an image processing engine, a storage module, a power source and an output module.

2. The optical apparatus of claim 1, wherein the output module is selected from the group of output modules consisting of a wireless output module, a NTSC output module, and a USB output module.

3. The optical apparatus of claim 1, further comprising a light-emitting diode within the removable cartridge, wherein the optical apparatus further comprises an optical couple for carrying light from a light source at least partially through the shaft.

4. The optical apparatus of claim 1, further comprising an accelerometer in the removable cartridge and in communication with the image processing engine, the accelerometer configured to cause the image processing engine to rotate an image from the camera in response to the accelerometer sensing rotation of the camera assembly.

5. The optical apparatus of claim 1, wherein the shaft is shapeable.

6. A method of ob-gyn examination comprising:
inserting a shaft of an optical apparatus into the vaginal canal of a patient;
rotating a camera assembly coupled to a distal end of the shaft radially relative to the shaft to expose a working channel, wherein the camera assembly comprises a camera;
displaying at least one image from the camera to allow for visual examination of at least one of the tissue and vascular structure of one or more of the cervix, the vagina, and the vulva.

7. The method of claim 6, further comprising inserting at least one surgical instrument into the vaginal canal through the working channel.

8. The method of claim 6, wherein said rotating further exposes light from a light-emitting diode.

9. The method of claim 8, wherein the method further comprises turning on the light-emitting diode in response to said rotating.

10. The method of claim 7, further comprising correcting the at least one image for the rotating prior to the displaying.

11. A method of ob-gyn examination comprising:
inserting a shaft of an optical apparatus into the vaginal canal of a patient;
capturing at least one image with a camera coupled to a distal end of the shaft without the use of fiber optics; and
moving, without rotating, the camera relative to the shaft to change the field of view of the camera.

12. The method of claim 11, wherein said moving, without rotating, the camera comprises moving the camera relative to the shaft about a hinge, wherein the hinge comprises an axis that is perpendicular to an axis of the shaft.

13. The method of claim 11, wherein said moving, without rotating, the camera comprises sliding the camera outwardly along a ramp interface between the camera and the distal end of the shaft.

14. The method of claim 11, wherein said moving, without rotating, the camera comprises:
coupling an arm of the camera to a rail formed in the shaft; and
sliding the arm through a predetermined path defined by the rail to move the camera outwardly from shaft.

15. An optical apparatus comprising:
a shaft having a first end and a second end and a working channel extending longitudinally through at least a portion of the shaft to the second end;
a handle coupled to the first end of the shaft;
a camera assembly comprising a camera;
a flexible circuit coupled to the camera and to the second end of the shaft;
a control member coupled to the camera assembly and to the second end of the shaft, wherein rotation of the pin causes rotation of the camera assembly relative to the shaft from a first configuration in which the camera assembly blocks an opening of the working channel to a second, rotated configuration in which the working channel is exposed.

16. The optical apparatus of claim 15, wherein the control member comprises a pin with a longitudinal axis that is parallel to a longitudinal axis of the shaft.

17. The optical apparatus of claim 15, wherein the control member extends to at least a proximal end of the shaft.

18. The optical apparatus of claim 15, wherein in the first configuration the camera assembly is aligned concentrically with the shaft and in the second configuration the camera assembly is spaced radially from the shaft.

19. The optical apparatus of claim 15, wherein the shaft further comprises a generally crescent-shaped lumen positioned concentrically with respect to the control member, the optical apparatus further comprising a second member configured to traverse the crescent-shaped lumen responsive to rotation of the control member coupled to the camera assembly.

20. The optical apparatus of claim 19, wherein the flexible circuit is positioned at least partially within the crescent-shaped lumen such the flexible circuit remains generally adjacent to the second member during the translation of the second member.

21. The optical apparatus of claim 15, wherein:
the camera assembly comprises a proximal circumferential step; and
the shaft comprises a circumferential step included on the distal end of the shaft,
wherein in the first configuration the circumferential step of the camera assembly is configured to mate with the circumferential step of the shaft and in the second configuration the circumferential step of the camera assembly is adapted to contact an outer surface of the shaft.

22. An optical apparatus comprising:
a shaft having a first end and a second end;
a handle coupled to the first end of the shaft;
a camera assembly comprising a camera; and
a flexible circuit extending between the camera assembly and to the second end of the shaft,
wherein the shaft and the camera assembly are coupled by a hinge joint and the flexible circuit extends through the hinge joint.

23. The optical apparatus of claim 22, wherein the hinge joint includes a cylindrical projection that is received by, and secured in place by frictional forces from, a cylindrical socket, and wherein a lumen for receiving the flexible circuit extends through the cylindrical projection.

24. The optical apparatus of claim 23, wherein the lumen has a tapered opening on the cylindrical projection.

25. The optical apparatus of claim 22, further comprising a hinge pin that extends laterally through the hinge joint.
